SEQUENCE LISTING (10 sequences)

SEQ ID NO: 1 - DNA, 1713 bp, Homo sapiens
  CDS (241)..(1134)
gcgcgagtgctcccgggaactctgcctgcgcggcggcagcgaccggaggccaggcccagcacgccggagctggcctgctggggaggggcgggaggcgcgcgcgggagggtccgcccggccaggccccgggccctcgcagaggccggccgcgctcccagcccgcccggagcccatgcccggcggctggccagtgctgcggcagaagggggggcccggctctgcatggccccggctgctgacatgacttctttgccactcggtgtcaaagtggaggactccgccttcggcaagccggcggggggaggcgcgggccaggcccccagcgccgccgcggccacggcagccgccatgggcgcggacgaggagggggccaagcccaaagtgtccccttcgctcctgcccttcagcgtggaggcgctcatggccgaccacaggaagccgggggccaaggagagcgccctggcgccctccgagggcgtgcaggcggcgggtggctcggcgcagccactgggcgtcccgccggggtcgctgggagccccggacgcgccctcttcgccgcggccgctcggccatttctcggtggggggactcctcaagctgccagaagatgcgctcgtcaaagccgagagccccgagaagcccgagaggaccccgtggatgcagagcccccgcttctccccgccgccggccaggcggctgagccccccagcctgcaccctccgcaaacacaagacgaaccgtaagccgcggacgcccttcaccaccgcgcagctgctggcgctggagcgcaagttccgccagaagcagtacctgtccatcgccgagcgcgcggagttctccagctcgctcagcctcactgagacgcaggtgaagatatggttccagaaccgccgcgccaaggcaaagagactacaagaggcagagctggagaagctgaagatggccgccaagcccatgctgccaccggctgccttcggcctctccttccctctcggcggccccgcagctgtagcggccgcggcgggtgcctcgctctacggtgcctctggccccttccagcgcgccgcgctgcctgtggcgcccgtgggactctacacggcccatgtgggctacagcatgtaccacctgacatagagggtcccaggtccccacctgtgggccagccgattcctccagccctggtgctgtacccccgacgtgctcccctgctcggcaccgccagccgccttccctttaaccctcacactgctccagtttcacctctttgctccctgagttcactctccgaagtctgatccctgccaaaaagtggctggaagagtcccttagtactcttctagcatttagatctacactctcgagttaaagatggggaaactgagggcagagaggttaacagatttatctagggtccccagcagaattgacagttgaacagagctagaggccatgtctcctgcatagcttttccctgtcctgacaccaggcaagaaaagcgcagagaaatcggtgtctgacgattttggaaatgagaacaatctcaaaaaaaaaaaaaaaaaaaaaaaaaaaagaaaagagaaaaaaaagactagccagccaggaagatgaatcctagcttcttccattggaaaatttaagacaagttcaacaacaaaacatttgctctggggggcagggaaaacacagatgtgttgcaaaggtaggttgaaggga

SEQ ID NO: 2 - protein, 297 aa, Homo sapiens
MTSLPLGVKVEDSAFGKPAGGGAGQAPSAAAATAAAMGADEEGAKPKVSPSLLPFSVEALMADHRKPGAKESALAPSEGVQAAGGSAQPLGVPPGSLGAPDAPSSPRPLGHFSVGGLLKLPEDALVKAESPEKPERTPWMQSPRFSPPPARRLSPPACTLRKHKTNRKPRTPFTTAQLLALERKFRQKQYLSIAERAEFSSSLSLTETQVKIWFQNRRAKAKRLQEAELEKLKMAAKPMLPPAAFGLSFPLGGPAAVAAAAGASLYGASGPFQRAALPVAPVGLYTAHVGYSMYHLT

SEQ ID NO: 3 - DNA, 2197 bp, Homo sapiens
  CDS (62)..(865)
gtcgccgctgccgggttgccagcggagtcgcgcgtcgggagctacgtagggcagagaagtcatggcttctccgtccaaaggcaatgacttgttttcgcccgacgaggagggcccagcagtggtggccggaccaggcccggggcctgggggcgccgagggggccgcggaggagcgccgcgtcaaggtctccagcctgcccttcagcgtggaggcgctcatgtccgacaagaagccgcccaaggaggcgtccccgctgccggccgaaagcgcctcggccggggccaccctgcggccactgctgctgtcggggcacggcgctcgggaagcgcacagccccgggccgctggtgaagcccttcgagaccgcctcggtcaagtcggaaaattcagaagatggagcggcgtggatgcaggaacccggccgatattcgccgccgccaagacatatgagccctaccacctgcaccctgaggaaacacaagaccaatcggaagccgcgcacgccctttaccacatcccagctcctcgccctggagcgcaagttccgtcagaaacagtacctctccattgcagagcgtgcagagttctccagctctctgaacctcacagagacccaggtcaaaatctggttccagaaccgaagggccaaggcgaaaagactgcaggaggcagaactggaaaagctgaaaatggctgcaaaacctatgctgccctccagcttcagtctccctttccccatcagctcgcccctgcaggcagcgtccatatatggagcatcctacccgttccatagacctgtgcttcccatcccgcctgtgggactctatgccacgccagtgggatatggcatgtaccacctgtcctaaggaagaccagatcaatagactccatgatggatgcttgtttcaaagggtttcctctccctctccacgaaggcagtaccagccagtactcctgctctgctaaccctgcgtgcaccaccctaagcggctaggctgacagggccacacgacatagctgaaatttgttctgtaggcggaggcaccaagccctgttttcttggtgtaatcttccagatgcccccttttcctttcacaaagattggctctgatggtttttatgtataaatatatatatataataaaatataatacatttttatacagcagacgtaaaaattcaaattattttaaaaggcaaaatttatatacatatgtgctttttttctatatctcaccttcccaaaagacactgtgtaagtccatttgttgtattttcttaaagagggagacaaattatttgcaaaatgtgctaaagtcaatgatttttacgggattattgacttctgcttatggaaaacaaagaaacagacacaatgcacacagaaaatattagatatggagagattattcaaagtgaaggggacacatcatatttctgcattttacttgcattaaaagaaacctctttatatactacagttgttcctatctctcccccgccccccaccgccccaccacacacatatttttaaagtttttccttttttaagaatatttttgtaagaccaatacctgggatgagaagaatcctgagactgcctggaggtgaggtagaaaattagaaatacttcctaattcttctcaaggctgttggtaactttatttcagataattggagagtaaaatgttaaaacctgttgagaggaattgatggtttctgagaaatactaggtacattcatcctcacagattgcaaaggtgatttgggtgggggtttagtaattttctgcttaaaaaatgagtatcttgtaaccattacctatatgctaaatattcttgaacaattagtagatccagaaagaaaaaaaaatatgctttctctgtgtgtgtacctgttgtatgtcctaaacttattagaaaattttatatacttttttacatgttggggggcagaaggtaaagccatgttttgacttggtgaaaatgggattgtcaaacagcccattaagttccctggtatttcaccttcctgtccatctgtcccctccctccggtatacctttatccctttgaaagggtgcttgtacaatttgatatattttattgaagagttatctcttattctgaattaaattaagcatttgttttattgcagtaaagtttgtccaaactcacaattaaaaaaaaaaaaaaaaa

SEQ ID NO: 4 - protein, 267 aa, Homo sapiens
MASPSKGNDLFSPDEEGPAVVAGPGPGPGGAEGAAEERRVKVSSLPFSVEALMSDKKPPKEASPLPAESASAGATLRPLLLSGHGAREAHSPGPLVKPFETASVKSENSEDGAAWMQEPGRYSPPPRHMSPTTCTLRKHKTNRKPRTPFTTSQLLALERKFRQKQYLSIAERAEFSSSLNLTETQVKIWFQNRRAKAKRLQEAELEKLKMAAKPMLPSSFSLPFPISSPLQAASIYGASYPFHRPVLPIPPVGLYATPVGYGMYHLS

SEQ ID NO: 5 - DNA, 2216 bp, Mus musculus
  CDS (97)..(711)
gaattccggcagagcccaaggtcctagcaaaataaaccgccgctgggtcccacgcgtcccagccgggcggctagccccgcacccgccatcccatgcatggcccgggcgacattcgacatgaacgcggcggggctcgaagctcgcggcgggggccacacagagcacggaccactccccttcagcgtcgagtctctgctggaggctgagcgcgtaccgggctccgagtctggggagctgggggtggagaggccgctcggcgcctcgaagcctggagcctggccccctccagtcgcgcactcttgtcccccacgtgccccgagccctccaccctgcaccctccgcaaacacaaaaccaatcgcaaaccacggacgccattcaccaccgcgcagctgctggcgcttgagcgcaagtttcaccagaagcaatacttatccattgcggagcgcgccgagttctccagcagcttgagcctcactgagactcaggtcaagatctggtttcagaaccgccgagccaaagccaagcggctgcaggaagctgagctggagaagctgaagctggcagcgaagccactactgccggcggcgtttgccctgcccttcccgctgggcacccagctgcacagctccgcggccactttcggcggcaacgcggttcctgggatcctcgccgggcctgtcgcggcctatggcatgtactacttgtcttagatcatggaatcaagccctaacctcttggttaggctcagggttgcgcacagttctgcctgccgctgatgctcctggtaacttttgacttttcattttagtcttccggggggggggggggggggggtttatacggtgttatacttgatgactggagcctatttatagacatcttacaaatactttaaaccttcctaaaaccttgcccacatcaatctgtttttctgaaagaaaacacaccgtcatatagtccttgaaacagacaaaagcagttcttcctatttcttacaaagcagtcgacaaagtatttctggagtgggtgtgctccctgtattcactctgcccggacaccgaggacttcagagaggctcgattgagctggcataagaaaacaaattccataagaagaaatgaaaataaaaaatgagataatcggacggttagaaattactgtctctccaactgttgtgaccaaatggaaacttttacgctttttaggttttataccattattatttttttttttagagtatatgtcgttcaaggagctagaggcaaacgctggatggacttggaagtaggggaggggactgttggcgccatgccctaatgtaagggtggctgctgcccccagcatctcctgagcttcttcgaaaagagcctgctggggggagacagagggctgaaggcagcagggaccaacctctagcctcttgccagcaggtgtccagtcaataagagaagaaagtgagagttgagcatctcagatctcccattgtgggtgcggtaggaggtgaggcctgtgaaggtcccagaagagttactctgagtgccggctgcctctgtaattctggtgctccgtagaaaagcggaggacgttgtttaggaatccggacttgtacctttattctacctttatccgaggaaagaaaacaggccatctgagctgttctgtcctatcctgttcggtctcaatcctatcatgcaagattgaggcccttagggtacctgacaacgggcactaatacaccccatggctgtttccacaggacacttttgctgcaatctagagactttagctggagtgtgttgagggctcacaacagtcccctggagtcacagcccaggacctttgacaggccctcctgccagccttcgggcaggtgaggaagaacctgcctgcagagggctgctggttgtgtgttcatgagaacatggaatttcacagtgatagatggcccagggttaggatgctctggtggggggaagggaaggactgggctatctcggcagtccaagccacgtccaggaaaactgctgtcccgtgaccttcactggtccatcagctccaaagtaaaaccgaaaagctaatcttgcatggaattgcctattgatacgtattgacagtcttctgccctgtgtctcttggaaaaattggaaaccagataggtttggttttgttttgagaccaaatcgtcactatgtagggggggggggggggggaattc

SEQ ID NO: 6 - protein, 204 aa, Mus musculus
MARATFDMNAAGLEARGGGHTEHGPLPFSVESLLEAERVPGSESGELGVERPLGASKPGAWPPPVAHSCPPRAPSPPPCTLRKHKTNRKPRTPFTTAQLLALERKFHQKQYLSIAERAEFSSSLSLTETQVKIWFQNRRAKAKRLQEAELEKLKLAAKPLLPAAFALPFPLGTQLHSSAATFGGNAVPGILAGPVAAYGMYYLS

SEQ ID NO: 7 - DNA, 1562 bp, Homo sapiens
  CDS (222)..(1370)
gtgaaatcagatcagccagagcagttcgctgtgactgatctctcctcccaccctacattctcttggctggaccctatcctcctggctgattctggtcgccctggacactccctcagttctttcccaggagtgcggtggctgctggcgccgagtcccagcgggcacggacgtcagacgcatcgtttcttctcctctacaggtcctcccggcccggcccgaacatgctggacggcctaaagatggaggagaacttccaaagcgcgatcgacacctcggcctccttctcctcgctgctgggcagagcggtgagccccaagtctgtctgcgagggctgtcagcgggtcatcttggacaggtttctgctgcggctcaacgacagcttctggcatgagcagtgcgtgcagtgcgcctcctgcaaagagcccctggagaccacctgcttctaccgggacaagaagctgtactgcaagtatgactacgagaagctgtttgctgttaaatgtgggggctgcttcgaggccatcgctcccaatgagtttgttatgcgggcccagaagagtgtataccacctgagctgcttctgctgctgtgtctgcgagcgacagcttcagaagggtgatgagtttgtcctgaaggaggggcagctgctctgcaaaggggactatgagaaggagcgggagctgctcagcctggtgagcccagcagcctcagactcaggtaaaagtgatgatgaagaaagtctctgcaagtcagcccatggggcagggaaaggaactgctgaggaaggcaaggaccataagcgccccaaacgtccgagaaccatcttgacaactcaacagaggcgagcattcaaggcctcatttgaagtatcctccaagccctgcaggaaggtgagagagactctggctgcagagacagggctgagtgtccgtgtcgtccaggtgtggttccaaaaccagagagcgaagatgaagaagctggccaggcgacagcagcagcagcagcaagatcagcagaacacccagaggctgagctctgctcagacaaacggtggtgggagtgctgggatggaaggaatcatgaacccctacacggctctgcccaccccacagcagctcctggccatcgagcagagtgtctacagctcagatcccttccgacagggtctcaccccaccccagatgcctggagaccacatgcacccttatggtgccgagccccttttccatgacctggatagcgacgacacctccctcagtaacctgggtgattgtttcctagcaacctcagaagctgggcctctgcagtccagagtgggaaaccccattgaccatctgtactccatgcagaattcttacttcacatcttgagtcttcccctagagttctgtgactaggctcccatatggaacaaccatattctttgaggggtcactggctttaggacagggaggccagggaagaggtgggttggggagggagttttgttggggatgctgttgtataatgatatggtgtagctcagcatttccaaagactgaatacattatggattgcatagtt

SEQ ID NO: 8 - protein, 382 aa, Homo sapiens
MLDGLKMEENFQSAIDTSASFSSLLGRAVSPKSVCEGCQRVILDRFLLRLNDSFWHEQCVQCASCKEPLETTCFYRDKKLYCKYDYEKLFAVKCGGCFEAIAPNEFVMRAQKSVYHLSCFCCCVCERQLQKGDEFVLKEGQLLCKGDYEKERELLSLVSPAASDSGKSDDEESLCKSAHGAGKGTAEEGKDHKRPKRPRTILTTQQRRAFKASFEVSSKPCRKVRETLAAETGLSVRVVQVWFQNQRAKMKKLARRQQQQQQDQQNTQRLSSAQTNGGGSAGMEGIMNPYTALPTPQQLLAIEQSVYSSDPFRQGLTPPQMPGDHMHPYGAEPLFHDLDSDDTSLSNLGDCFLATSEAGPLQSRVGNPIDHLYSMQNSYFTS

SEQ ID NO: 9 - DNA, 1119 bp, Homo sapiens
  CDS (1)..(1119)
atgttggacggcatcaagatggaggagcacgccctgcgccccgggcccgccactctgggggtgctgctgggctccgactgcccgcatcccgccgtctgcgagggctgccagcggcccatctccgaccgcttcctgatgcgagtcaacgagtcgtcctggcacgaggagtgtttgcagtgcgcggcgtgtcagcaagccctcaccaccagctgctacttccgggatcggaaactgtactgcaaacaagactaccaacagctcttcgcggccaagtgcagcggctgcatggagaagatcgcccccaccgagttcgtgatgcgggcgctggagtgcgtgtaccacctgggctgcttctgctgctgcgtgtgtgaacggcagctacgcaagggcgacgaattcgtgctcaaggagggccagctgctgtgcaagggtgactacgagaaggagaaggacctgctcagctccgtgagccccgacgagtccgactccgtgaagagcgaggatgaagatggggacatgaagccggccaaggggcagggcagtcagagcaagggcagcggggatgacgggaaggacccgcggaggcccaagcgaccccggaccatcctcaccacgcagcagcgaagagccttcaaggcctccttcgaggtctcgtcgaagccttgccgaaaggtccgagagacactggcagctgagacgggcctcagtgtgcgcgtggtccaggtctggtttcagaaccaaagagcaaagatgaagaagctggcgcggcggcaccagcagcagcaggagcagcagaactcccagcggctgggccaggaggtcctgtccagccgcatggagggcatgatggcttcctacacgccgctggccccaccacagcagcagatcgtggccatggaacagagcccctacggcagcagcgaccccttccagcagggcctcacgccgccccaaatgccagggaacgactccatcttccatgacatcgacagcgatacctccttaaccagcctcagcgactgcttcctcggctcctcagacgtgggctccctgcaggcccgcgtggggaaccccatcgaccggctctactccatgcagagttcctacttcgcctcctga

SEQ ID NO: 10 - protein, 372 aa, Homo sapiens
MLDGIKMEEHALRPGPATLGVLLGSDCPHPAVCEGCQRPISDRFLMRVNESSWHEECLQCAACQQALTTSCYFRDRKLYCKQDYQQLFAAKCSGCMEKIAPTEFVMRALECVYHLGCFCCCVCERQLRKGDEFVLKEGQLLCKGDYEKEKDLLSSVSPDESDSVKSEDEDGDMKPAKGQGSQSKGSGDDGKDPRRPKRPRTILTTQQRRAFKASFEVSSKPCRKVRETLAAETGLSVRVVQVWFQNQRAKMKKLARRHQQQQEQQNSQRLGQEVLSSRMEGMMASYTPLAPPQQQIVAMEQSPYGSSDPFQQGLTPPQMPGNDSIFHDIDSDTSLTSLSDCFLGSSDVGSLQARVGNPIDRLYSMQSSYFAS

(19) United States
(12) Patent Application Publication
(10) Pub. No.: US 2008/0311091 A1
(43) Pub. Date: Dec. 18, 2008

(54) Engineered Dopamine Neurons and Uses Thereof

(76) Inventors: Thomas Perlmann, Stockholm (SE); Johan Ericson, Stockholm (SE); Elisabet Andersson, Stockholm (SE)

Correspondence Address: WOLF GREENFIELD & SACKS, P.C., 600 ATLANTIC AVENUE, BOSTON, MA, 02210-2206, US

(21) Appl. No.: 11/793,797

(22) PCT Filed: Dec. 22, 2005
(86) PCT No.: PCT/US2005/046673

§ 371 Date: May 19, 2008

Related U.S. Application Data
(60) Provisional application No. 60/638,951, filed on Dec. 23, 2004.

(51) Int. Cl. A61K 48/00; C12N 5/10; C12N 5/02; A61K 35/30; C40B 30/06; C12Q 1/68; A61P 25/16

(52) U.S. Cl. 424/93.21; 435/368; 435/366; 435/377; 506/10; 453/6

(57) ABSTRACT

The invention relates to dopamine neuron determinants, the use of these determinants in differentiating cells to dopamine neurons, cells produced by the over-expression of these determinants, and uses of these cells.

### RELATED APPLICATIONS

This application claims the benefit under 35 U.S.C. § 119(e) of U.S. provisional patent application Ser. No. 60/638,951, filed Dec. 23, 2004, the contents of which are incorporated herein by reference in their entirety.

### FIELD OF THE INVENTION

The invention relates to dopamine neuron determinants, the use of these determinants in differentiating cells to dopamine neurons, cells produced by the over-expression of these determinants, and uses of these cells.

### BACKGROUND OF THE INVENTION

Most classes of neurons that constitute the central nervous system (CNS) are generated during embryonic development in response to local inductive cues. Such signals act to regulate gene expression in responding neural progenitor cells, which eventually emerge in the generation of specific neuronal subtypes at different positions of the developing central nervous system (Jessell, 2000). Mesencephalic dopamine (DA) neurons that degenerate in patients with Parkinson's disease, are derived from progenitor cells located at the ventral midline of the midbrain while progenitors at adjacent lateral positions generate motor neurons or different subtypes of interneurons (Hynes and Rosenthal, 1999). Characterization of the molecular program that controls the normal generation of DA neurons during embryonic development is likely to facilitate the engineering of DA neurons from stem cells which, in turn, may be critical for the development of stem cell-based therapies of Parkinson's disease.

Factors that control patterning of the ventral midbrain are poorly defined and the molecular pathway underlying the emergence of DA neurons remains obscure. The generation of DA neurons depend on sonic hedgehog (Shh) signalling provided by ventral midline cells and on the activity of the fibroblast growth factor (FGF) family member 8 (FGF8), secreted by the isthmic organizer (Hynes et al., 1995a; Hynes et al., 1995b). The primary role for these signaling molecules is to establish a ventral midbrain identity at initial stages of neural development. Several previously characterized transcription factors, including Nurr1, Pitx3, Engrailed and Lmx1b, are involved in the maturation of postmitotic DA neurons but not in the initial specification of these cells (Nunes et al., 2003; Simon et al., 1998; Smidt et al., 1997; van den Munckhof et al., 2003; Wallen and Perlmann, 2003; Zetterström et al., 1997). Thus, transcriptional DA cell determinants functioning downstream of Shh and upstream of transcription factors that control the postmitotic differentiation of DA neurons should exist but have not yet been identified.

A unique property of DA neurons is their generation from the midline of the ventral mibrain. The ventral midline of the neural tube is initially occupied by Shh-expressing glial-like floor plate cells (Placzek and Briscoe, 2005; Placzek et al., 1993; Placzek et al., 1990). Thus, in contrast to most other ventral neuronal subtypes, the generation of DA neurons must be preceded by a conversion of floor plate cells into neuronal progenitor cells. How such a conversion occurs, and the identity of factors controlling the transition, are other unresolved questions.

Studies of the developing spinal cord have provided insights into basic strategies of neuronal cell fate specification (Jessell, 2000). At this axial level, Shh controls the spatially restricted expression of a set of homeodomain (HD) transcription factors in ventral progenitor cells (Briscoe et al., 2000). These HD proteins, in turn, control the positional generation of motor neurons and interneurons by triggering the activation of specific downstream genes that establish the subtype identity of postmitotic neurons (Briscoe et al., 2000; Jessell, 2000; Lee and Pfaff, 2001; Muhr et al., 2001; Pierani et al., 2001). It seems likely that midbrain DA neurons are specified by similar mechanisms.

DA neurons of the VMB degenerate in patients with Parkinson's disease. Although adult DA neurons are affected, the identification of the inductive cues and cell intrinsic transcriptional determinants that underlie the normal generation of DA neurons is important and needed for several reasons. First, such studies are likely to uncover factors that contribute to the maintenance and repair also of mature DA neurons. Second, better protocols for generating DA neurons from stem cells will require a clearer understanding of how DA neurons are specified. Ultimately, such protocols will help both in the development of cell replacement therapies and will also provide more efficient tools for the identification of additional factors contributing to the development, survival and maintenance of the dopaminergic system.

Previous studies have shown that Parkinson's disease patients can be treated by transplantation of fetal dopamine (DA) neurons. The limited source of human fetal DA neurons, and complicated ethical considerations, makes the use of fetal cell transplants unlikely as a future treatment of Parkinson's disease patients. In contrast, an unlimited source of DA neurons generated from stem cells and other neural or neuronal precursors or progenitors would provide a highly attractive alternative. However, this requires efficient methods whereby these cells are engineered in vitro, which are not currently available. Therefore, there is a need to identify DA neuron determinants, which can be used for preparation of DA neurons useful in the treatment of Parkinson's disease and other disease in which there is degeneration of DA neurons.

### SUMMARY OF THE INVENTION

We have identified homeodomain (HD) containing transcription factors, Msx1, Msx2, Msx3, Lmx1a and Lmx1b, that are critically involved in DA neuron development in vivo and thus are dopamine neuron determinants. Importantly, when the Msx1, Msx2 and Lmx1a genes are expressed in mouse embryonic stem cells, either alone or in combination, they induce robust differentiation into DA neurons. The Msx3 and Lmx1b genes are highly homologous to Msx1/2 and Lmx1a genes, and therefore are expected to have similar function in differentiating cells to DA neurons.

This new method for DA neuron engineering provides methods whereby unlimited numbers of DA neurons can be generated for transplantation in Parkinson's disease patients and patients having other disorders where there is degeneration of DA neurons, for drug screening in vitro, etc.

According to one aspect of the invention, a cell that over-expresses Msx1, Msx2, Msx3, Lmx1a and/or Lmx1b is provided. The cell, when cultured, differentiates into a DA neuron. According to another aspect of the invention, a dopamine (DA) neuron differentiated from a cell by over-expression of Msx1, Msx2, Msx3, Lmx1a and/or Lmx1b in the cell is provided. Preferably the cell or DA neuron is isolated. In some embodiments, the cell is a stem cell or a non-neural, neural or neuronal progenitor or precursor; a preferred stem cell is an embryonic stem cell.

In certain embodiments, the Msx1, Msx2, Msx3, Lmx1a and/or Lmx1b are expressed recombinantly. Preferably, the recombinant expression is effectuated by operably linking the Msx1, Msx2, Msx3, Lmx1a and/or Lmx1b genes to an exogenous promoter and/or enhancer sequence. In some of these preferred embodiments, the exogenous promoter and/or enhancer sequence is contained in an expression vector that is introduced into the cell; the expression vector in various embodiments is introduced by transfection, infection, microinjection, electroporation or recombination. Preferred promoters and enhancers include the promoter from the herpes simplex virus thymidine kinase gene, the promoter from the Rous sarcoma virus LTR, the enhancer from the nestin gene, and the Sonic hedgehog (Shh) enhancer.

In all of these embodiments, the preferred cell or DA neuron is human.

Preferably the Msx1, Msx2, Msx3, Lmx1a and/or Lmx1b is encoded by a nucleic acid molecule comprising SEQ ID NO:1 encoding Msx1, SEQ ID NO:3 encoding Msx2, SEQ ID NO:5 encoding Msx3, SEQ ID NO:7 encoding Lmx1a, SEQ ID NO:9 encoding Lmx1b, a coding sequence thereof, or a fragment or derivative thereof that contributes to inducing differentiation of the cell into a DA neuron.

Also provided are cell lines comprising the foregoing cells or DA neurons.

In still another aspect of the invention, isolated population of cells or DA neurons are provided. The isolated population optionally includes a carrier, preferably a pharmaceutically acceptable carrier and/or a cell freezing medium or cell storage medium.

Cultures of the foregoing cells and/or DA neurons also are provided.

According to another aspect of the invention, methods for producing dopamine (DA) neurons are provided. The methods include increasing the expression of Msx1, Msx2, Msx3, Lmx1a and/or Lmx1b in a cell, and culturing the cell having increased expression of Msx1, Msx2, Msx3, Lmx1a and/or Lmx1b under conditions and for a time sufficient to permit differentiation of the cell to a DA neuron. In some embodiments, the cell is isolated. In certain embodiments, the methods also include isolating the DA neuron.

In other embodiments, the cell is a stem cell or a non-neural, neural or neuronal progenitor or precursor; a preferred stem cell is an embryonic stem cell.

In certain embodiments, the expression of Msx1, Msx2, Msx3, Lmx1a and/or Lmx1b is increased by over-expressing one or more nucleic acids that encode Msx1, Msx2, Msx3, Lmx1a and/or Lmx1b proteins, preferably by expressing the nucleic acids recombinantly. Preferably, the recombinant expression is effectuated by operably linking the Msx1, Msx2, Msx3, Lmx1a and/or Lmx1b genes to an exogenous promoter and/or enhancer sequence. In some of these preferred embodiments, the exogenous promoter and/or enhancer sequence is contained in an expression vector that is introduced into the cell; the expression vector in various embodiments is introduced by transfection, infection, microinjection, electroporation or recombination. Preferred promoters and enhancers include the promoter from the herpes simplex virus thymidine kinase gene, the promoter from the Rous sarcoma virus LTR, the enhancer from the nestin gene, and the Sonic hedgehog (Shh) enhancer.

In all of these methods, the preferred cell or DA neuron is human.

Preferably the Msx1, Msx2, Msx3, Lmx1a and/or Lmx1b is encoded by a nucleic acid molecule comprising SEQ ID NO:1 encoding Msx1, SEQ ID NO:3 encoding Msx2, SEQ ID NO:5 encoding Msx3, SEQ ID NO:7 encoding Lmx1a, SEQ ID NO:9 encoding Lmx1b, a coding sequence thereof, or a fragment or derivative thereof that contributes to inducing differentiation of the cell into a DA neuron.

In various embodiments, the step of culturing is performed in vitro, in vivo, or ex vivo.

According to a further aspect of the invention, methods for differentiating a cell to a dopamine neuron in vivo are provided. The methods include ectopically expressing a dopamine neuron determinant in the cell, preferably by administering to a subject an expression vector that expresses the dopamine neuron determinant.

In some embodiments, the cell is an adult neural stem cell. The dopamine neuron determinant preferably is Msx1, Msx2, Msx3, Lmx1a and/or Lmx1b. In certain embodiments, the subject has or is suspected of having Parkinson's disease.

According to still another aspect of the invention, methods for cell transplantation are provided. The methods include obtaining the foregoing dopamine (DA) neurons, and transplanting the DA neurons into a subject. Preferably the DA neurons are transplanted into the brain of the subject, more preferably into the striatum.

In another aspect of the invention, other methods for cell transplantation are provided. The methods include obtaining dopamine (DA) neurons produced by the foregoing methods, and transplanting the DA neurons into a subject. Preferably the DA neurons are transplanted into the brain of the subject, more preferably into the striatum.

According to yet another aspect of the invention, methods for treating Parkinson's disease are provided. The methods include obtaining the foregoing dopamine (DA) neurons, and transplanting the DA neurons into a subject having or suspected of having Parkinson's disease. Preferably the DA neurons are transplanted into the brain of the subject, more preferably into the striatum.

According to still another aspect of the invention, methods for treating Parkinson's disease are provided. The methods include obtaining dopamine (DA) neurons produced by the foregoing methods, and transplanting the DA neurons into a subject having or suspected of having Parkinson's disease. Preferably the DA neurons are transplanted into the brain of the subject, more preferably into the striatum.

In another aspect of the invention, methods for identifying compounds useful in the differentiation of stem cells, neural and/or neuronal progenitors or precursors to dopamine (DA) neurons are provided. The methods include contacting stem cells, neural and/or neuronal progenitors or precursors with a candidate compound under conditions that, in the absence of the candidate compound, result in a baseline amount of expression of Msx1, Msx2, Msx3, Lmx1a and/or Lmx1b; and determining a test amount of expression of the Msx1, Msx2, Msx3, Lmx1a and/or Lmx1b in the presence of the candidate compound as a measure of the effect of the compound. A test amount of expression of the Msx1, Msx2, Msx3, Lmx1a and/or Lmx1b that is greater than the baseline amount indicates that the candidate compound is a compound that is useful in the differentiation of stem cells, neural and/or neuronal progenitors or precursors to DA neurons.

In certain embodiments, the compound is a set of compounds in a library of molecules. Preferably the library is a natural product library, a library generated by combinatorial chemistry, or a library of known drug molecules.

Methods for identifying compounds useful in modulating behavior of dopamine (DA) neurons are provided in another aspect of the invention. The methods include contacting the foregoing DA neurons with a candidate compound under conditions that, in the absence of the candidate compound, result in a baseline amount of behavior of the DA neurons; and determining a test amount of behavior of the DA neurons in the presence of the candidate compound as a measure of the effect of the compound. A test amount of behavior of the DA neurons that is greater than the baseline amount indicates that the candidate compound is a compound that is useful in modulating the behavior of DA neurons.

In certain embodiments, the compound is a set of compounds in a library of molecules. Preferably the library is a natural product library, a library generated by combinatorial chemistry, or a library of known drug molecules.

In preferred embodiment, the modulation of the behavior of DA neurons is increasing the growth and/or survival of DA neurons, increasing dopamine synthesis, increasing dopamine storage, or increasing dopamine release.

According to another aspect of the invention, methods for identifying DA neuron progenitor cells are provided. The methods include determining the expression in a cell of one or more Lmx1a, Msx1 and/or Msx2 gene products. The expression of the one or more gene products indicates that the cell is a DA neuron progenitor cell.

In some embodiments, the DA neuron progenitor cells are embryonic progenitor cells. In other embodiments, the one or more gene products is RNA. In the latter case, the expression of the one or more gene products preferably is determined by RT-PCR or by nucleic acid hybridization. In further embodiments, the one or more gene products is a protein. In such embodiments, the expression of the one or more gene products is determined by binding of an antibody or antibody fragment to the protein.

In preferred embodiments, the one or more gene products includes one or more Lmx1a gene products, or one or more Msx1 gene products, or one or more Msx2 gene products.

According to another aspect of the invention, a DA neuron progenitor cell identified by any of the foregoing methods is provided.

According to still another aspect of the invention, methods for isolating DA neuron progenitor cells are provided. The methods include contacting a population of cells with a reagent that binds to of one or more Lmx1a, Msx1 and/or Msx2 gene products, and isolating cells that are bound by the reagent from the population. In preferred embodiments, the DA neuron progenitor cells are embryonic progenitor cells, the reagent is labeled, and/or the one or more gene products is a protein, in which case the reagent is preferably an antibody or binding fragment thereof.

In other embodiments, the one or more gene products includes one or more Lmx1a gene products, or one or more Msx1 gene products, or one or more Msx2 gene products.

According to another aspect of the invention, a DA neuron progenitor cell, preferably an embryonic progenitor cell, isolated by any of the foregoing methods is provided. Also provided are DA neurons differentiated from these DA neuron progenitor cells.

According to yet another aspect of the invention, methods of treating a neurodegenerative disease or disorder are provided. The methods include obtaining DA neurons or progenitor cells isolated or differentiated by the foregoing methods, and transplanting the DA neurons or progenitor cells into a subject having or suspected of having the neurodegenerative disease or disorder. In preferred embodiments, the neurodegenerative disease or disorder is Parkinson's disease. In other preferred embodiments, the DA neurons or progenitor cells are transplanted into the brain of the subject, more preferably into the striatum.

According to another aspect of the invention, additional methods for isolating DA neuron progenitor cells and/or DA neurons are provided. The methods include providing a population of cells that comprises a nucleic acid molecule that encodes a marker protein operatively linked to a Lmx1a, Msx1 and/or Msx2 promoter sequence, and isolating cells that express the marker protein from the population.

In some embodiments, the DA neuron progenitor cells are embryonic progenitor cells. In other embodiments, the marker protein is a fluorescent protein, preferably a green fluorescent protein, or a cell surface protein. In further embodiments, the cells that express the marker protein are isolated by fluorescence activated cell sorting or magnetic sorting. The magnetic sorting preferably includes contacting the population of cells with an antibody or binding fragment thereof that binds to the marker protein, wherein the antibody or binding fragment thereof is linked to a magnetic molecule or particle, and subjecting the population of cells to a magnetic field to separate cells bound by the antibody or binding fragment thereof.

In still other embodiments, the population of cells used in the foregoing methods includes stem cells, preferably embryonic stem cells, adult stem cells, orgenetically engineered stem cells. In other embodiments, the population of cells are genetically engineered cells.

DA neuron progenitor cells or DA neurons isolated by the foregoing methods also are provided, as are DA neurons differentiated from the DA neuron progenitor cells.

According to another aspect of the invention, methods of treating a neurodegenerative disease or disorder are provided. The methods include obtaining the foregoing dopamine neurons or progenitor cells, and transplanting the DA neurons or progenitor cells into a subject having or suspected of having the neurodegenerative disease or disorder. In preferred embodiments, the neurodegenerative disease or disorder is Parkinson's disease. In other preferred embodiments, the DA neurons or progenitor cells are transplanted into the brain of the subject, more preferably into the striatum.

The use of the foregoing products and methods in manufacturing a medicament for treatment of disease also is provided. Preferred diseases include neurodegenerative disease, particularly Parkinson's disease, and any other disease or disorder in which DA neurons or their activity are less than normal.

These and other aspects and embodiments of the invention will be described in further detail in connection with the detailed description of the invention.

### BRIEF DESCRIPTION OF THE DRAWINGS

FIG. 1: Expression of Lmx1a and Msx1 in ventral midbrain (vMB) of mouse embryos. Lmx1a (FIG. 1A) and Msx1 (FIG. 1B) whole mount in situ hybridization at E11.5 (vMB, yellow box). (FIG. 1C-F) Transverse sections through midbrain. Expression of Lmx1a and Msx1 relative to TH are shown (FIG. 1D, F) and Nurr1 (FIG. 1E) at E12.5. (FIG. 1G-V) Expression profiles of (FIG. 1G-J) Lmx1a and Msx1/2, (FIG. 1K-N) Lmx1a and Nkx6.1, (FIG. 1O-R) Msx1/2 and Nkx6.1, (FIG. 1S-V) Lmx1a and Lmx1b in vMB between E9 and E11.5.

FIG. 2: Shh induces expression of Msx1 and Lmx1a in vitro. (FIG. 2A) Schematic picture of a HH stage 6 chick embryo indicating axial levels of explants. (FIG. 2B-E) Transverse sections showing expression of (FIG. 2B) Lmx1a and Msx1/2 (FIG. 2C) Msx1/2 and Nurr1 (FIG. 2D) Lmx1a and Lmx1b (FIG. 2E) TH (arrowheads) and Nurr1 in chick vMB at HH stage 26. (FIG. 2F) Intermediate [i] control explants express Pax7+ but not Nkx2.2 or Msx1/2. (FIG. 2G) Midbrain [i]M explants grown in presence of 15 nM Shh express Nkx2.2, Msx1/2, Lmx1a, Nurr1 and TH but not Pax7. Shh-exposed explants from forebrain [i]F and hindbrain [i]H levels express Nkx2.2 but not Pax7, Msx1/2, Lmx1a, Nurr1 or TH. F, forebrain; M, midbrain; H, hindbrain; D, dorsal; V, ventral; A, anterior; P, posterior

FIG. 3: Lmx1a, but not Msx1, induces ectopic DA neurons in the chick midbrain. (FIG. 3A) Transverse sections through vMB of electroporated chick embryos. Forced expression of RCAS-Lmx1a, but not RCAS-eGFP or RCAS-Msx1, induces Nurr1+ and Lmx1b+ cells after 84 hours post transfection (hpt). Endogenous DA neuron domain encircled. (FIG. 3B-K) High magnification micrographs of vMB (red square in FIG. 3L). RCAS-Lmx1a, but not RCAS-eGFP, induces ectopic expression of Nurr1, Lmx1b (B, C) and TH (FIG. 3D, E) in post-mitotic neurons, and Msx1 in progenitors (FIG. 3F, G) and repressed Nkx6.1 expression (FIG. 3H, I). Induction of DA neurons by Lmx1a was accompanied by a reduction of Lim1+ interneurons (FIG. 3J, K). Embryos in FIG. 3B, C, J, K were harvested after 84 hpt, FIG. 3D, E after 110 hpt and FIG. 3F-I after 60 hpt. Dotted lines indicate border between ventricular zone (VZ) and marginal zone (MZ). (FIG. 3M-N) Quantification of Msx1, Nurr1 and Lmx1b cells induced by Lmx1a in ventral (FIG. 3M) and dorsal (FIG. 3N) progenitor cells. To calculate ventral Lmx1a induced cells, positive cells on the transfected side were subtracted by the number of cells on the control side. RCAS-Lmx1a did not induce DA neurons when expressed in the hindbrain and forebrain (data not shown).

FIG. 4: Lmx1a is required for generation of DA neurons. Chick embryos were transfected with a control siRNA or siRNA directed against Lmx1a mRNA and analyzed at 72 hpt. In controls, expression Lmx1a (FIG. 4A), Msx1/2 (FIG. 4C), Lmx1b (FIG. 4E), and Nurr1 (FIG. 4G), and motor neuron marker, Isl1/2 (FIG. 4I), were unaffected. In Lmx1a siRNA experiments, Lmx1a protein expression was significantly reduced (FIG. 4B) and Msx1/2 (FIG. 4D) and Nurr1 expression (FIG. 4H) were abolished. Lmx1b expression was lost in the marginal zone but not in progenitor cells (FIG. 4F). Dorsal expression of Lmx1a were unaffected in siRNA experiments (FIG. 4K, L). Dashed line indicate progenitor and marginal zone boundary.

FIG. 5: Msx1 represses Nkx6.1 expression. (FIG. 5A-B) Lmx1a and Msx1 fused to Gal4 were examined in luciferase reporter assays in COS-7 cells. Error bars indicate standard deviation, n=3. (FIG. 5A) The repressor activity of Msx1 was enhanced by the Grg4. (FIG. 5B) The activator function of Lmx1a was enhanced by Ldb1. (FIG. 5C-J) Forced expression of Msx1, Lmx1a and Msx1/Lmx1a in the chick midbrain. (FIG. 5C-F) Expression of pECE-Msx1 (FIG. 5D), but not pECE-Lmx1a (FIG. 5E) repressed Nkx6.1 after 20 hpt. (FIG. 5G-J) Forced expression of both Msx1 and Lmx1a repressed Nkx6.1 (FIG. 5F) at 20hpt and prematurely induced Nurr1+ cells (FIG. 5J) at 60 hpt. Expression of Msx1 (FIG. 5H) or Lmx1a (FIG. 5I) alone had not induced Nurr1 at 60hpt. Expression of eGFP (FIG. 5C, G) had no effect in these experiments. Dotted line indicates ventral midline.

FIG. 6: Msx1 induces Ngn2 expression and pan-neuronal differentiation. Figure shows transversesections through mouse vMB. Normal expression of Shh (A, B) and Ngn2 (C, D) at E9.5 and E11.5. Premature expression of Msx1 in ShhE-transgenic mice represses Nkx6.1 (E, F) at E9, induces Ngn2 (I, J) at E9.5, the pan-neuronal marker Nsg1 (K, L) and DA specific markers Nurr1 (M, N), Pitx3 (O, P) and TH (Q, R) at E10.5, but had no effects on Lmx1a induction (G, H). A premature loss of Shh expression was observed in ShhE-Msx1 transgenic mice (U, V). Isl1/2 expression was unaffected (S, T). Red brackets indicate DA progenitor domain boundaries. While Msx1-expression in ShhE-Msx1 transgenic embryos resulted in precocious production of DA neurons in all analyzed embryos (37/37), ectopic DA neurons and a reduction of Isl1/2+ motor neurons were observed in 3/37 embryos (data not shown). This weak ability of Msx1 to induce DA neurons at ectopic positions might depend on Lmx1b which is expressed in a relatively broad domain at early stages in mouse embryos (but not in the chick; see also FIG. 1S-U; data not shown).

FIG. 7: Lmx1a induces DA neurons from ES cells. FIG. 7 shows differentiated ES cells transfected with NesE-Lmx1a, NesE-eGFP or NesE-Lmx1b. (FIG. 7A) Expression of progenitor markers in ES cells grown in the presence or absence of Shh for four days. 1.7 nM Shh ventralizes cells as indicated by the loss of Pax7 and transfection of Lmx1a induces Msx1/2 expression. (FIG. 7B) After 8 days, Lmx1a, but not eGFP, induces TH+ neurons. (FIG. 7C) Lmx1a-induced TH+ cells co-express Nurr1, En, DAT, Pitx3, and Lmx1a but not GABA. (FIG. 7D) Diagram shows percentage of Lmx1a+ and Msx1+ colonies after 4 days, Nkx6.1+ colonies after 5 days and TH+ colonies after 8 days of differentiation with 1.7 nM Shh. Note that Lmx1b is extensively less effective to induce TH+ cells as compared to Lmx1a. (FIG. 7E) Diagram shows that >95% of Lmx1a-induced TH+ neurons co-express En1, Nurr1 and Pitx3 and <1% express GABA. ˜65% of TH+ cells generated after 12 days of culture in presence of 15 nM Shh co-express GABA. (FIG. 7F) Control ES cells differentiated for 8, 12 or 16 days in presence of 15 nM Shh. TH+ cells lack expression of Lmx1a, Lmx1b and DAT but co-express GABA. Lmx1a+ and Lmx1+ cells could be detected but these were distinct from TH+ cells. Lmx1a and Lmx1b expression were only rarely detected in the same cells (data not shown). Addition of 1.7, 3.7 or 7.5 nM Shh showed that lower concentrations of Shh were also unable to induce a correct DA neuron identity (data not shown). d=days of culture. (FIG. 7G) Model of DA neuron specification. Shh induces floor plate (FP) cells and Lmx1a. The induction of Lmx1a may be indirect. Lmx1a, in turn, induces Msx1 in DA progenitors and activates DA cell specific properties in differentiating cells. Msx1 suppresses Nkx6.1 and alternative cell fates, and induces Ngn2 expression. The Msx1-mediated induction of Ngn2 results in the suppression of FP characteristics and induction of panneuronal differentiation. Since Lmx1a only induces DA neurons in ventral progenitors it is likely that ongoing Shh signaling, or an Shh-induced activity, operates in parallel to Lmx1a.

FIG. 8: Msx1 induces Ngn2 in differentiating ES cells. FIG. 8 shows differentiated ES cells transfected with NesE-Msx1, NesE-Lmx1a, or NesE-eGFP. (FIG. 8A) Msx1 induces Ngn2 after 4 days but cannot induce TH+ cells after 8 days when differentiated in 1.7 nM Shh. (FIG. 8B) The diagram shows the percentage of Ngn2+ cells after 4 days of differentiation. (FIG. 8C) The diagram shows the percentage of TH+ colonies after 6 days of differentiation. Note that there is a synergistic effect between Msx1 and Lmx1a in the induction of TH+ neurons. An eGFP control is shown in FIG. 7D.

FIG. 9: (FIG. 9A) Mapping of presumptive forebrain (Otx1⁺En⁻Gbx2⁻), midbrain (Otx1⁺En⁺Gbx2⁻) and hindbrain (Otx1⁻En⁺Gbx2⁺) intermediate explants. (FIG. 9B) Exposure to BMP4 was also sufficient to induce Lmx1a and Msx1 in explants. However, BMP4 treatment of explants from all three axial levels leads to conversion into roof plate tissue expressing Msx1/2 and Lmx1a (24 hours), but not Nurr1 (72 hours). i, intermediate; F, forebrain; M, midbrain; H, hindbrain.

FIG. 10: Mouse Grg4 and Msx1 interact in vitro. (FIG. 10A) GST-Grg4 fusion protein interacts with 35S-labeled Msx1. (FIG. 10B) Removal of the Eh1 domain in Msx1 protein reduces the ability of GST-Grg4 fusion protein to interact with ³⁵S-labeled Msx1ΔEh1.

FIG. 11: Loss of Msx1 leads to a reduction in DA neurons at E11.5. The expression of Nkx6.1, Ngn2 and Nurr1 ws analysed in wild type and Msx1−/− embryos using immunohistochemistry. Note that there is an ectopic expression of Nkx6.1 and a reduction in the proneural protein Ngn2 and the DA marker Nurr1 in the DA progenitor domain of Msx1−/− embryos. The expression of Msx2 was not changed as shown by in situ hybridization which may explain the mild phenotype. The table shows the percent cells in Msx1−/− embryos compared to wt embryos counted on three consecutive sections from the anterior MB. Significance for the test was assumed at the level of p<0.05, n=4 (p<0.05*, p<0.01**, p<0.001*** etc).

FIG. 12: Lmx1a is expressed in essential all Shh+ cells in the ventral midbrain. FIG. 12 shows transverse section of mouse E10.75 vMB.

FIG. 13: Control (wt) embryonic stem (ES) cells or stable ES cell lines expressing Lmx1A (NesE-Lmx1a) or Msx1 (NesE-Msx 1) under control of a Nestin enhancer were cultured for eight (d8) or sixteen (d16) days in the presence of 7.5 nM Shh, 100 μg FGF8 and 2 μg FGF2. Growth factors were removed at day three of differentiation. After eight days of differentiation, 85-100% and 55-65% of Tuj 1⁺ neurons in Lmx1a- and Msx1-expressing ES cells co-expressed the dopamine neuron marker tyrosine hydroxylase (TH), respectively. Few TH⁺ neurons (<5%) could be detected in the wt control cells at this stage. After 16 days, essentially all TH⁺ neurons induced by Lmx1a and Msx1 co-expressed the midbrain dopamine neuron markers dopamine transporter (DAT) and Lmx1b. Very few TH⁺ cells cultures co-expressed these midbrain dopamine cell markers in control ES cell.

### DETAILED DESCRIPTION OF THE INVENTION

We have identified several genes that are critically involved in dopamine (DA) neuron development in vivo. These genes and their gene products can be used to differentiate stem cells, neural precursors and/or progenitors to DA neurons. These genes are referred to herein as “dopamine neuron determinants”, “DA neuron determinants” and the like.

Since the majority of cell intrinsic determinants that control cell decisions at spinal cord levels are homeodomain (HD) containing transcription factors (Jessell, 2000; Lee and Pfaff, 2001), we hypothesized that HD proteins are likely to be involved in the specification of DA neurons. To identify such factors, we applied a PCR-based approach to screen for HD proteins expressed in micro-dissected VMB tissue isolated from embryonic day 10.5 (E10.5) mouse embryos, a stage when DA neurons are being specified (Hynes and Rosenthal, 1999; Wallen and Perlmann, 2003). This resulted in the identification of the closely related HD proteins Msx1 and Msx2. Moreover, in a large scale in situ hybridization screen we also identified a third HD protein, Lmx1a, that is expressed in both DA progenitor cells and in differentiating DA neurons. Below we describe methods to induce DA neurons by forced expression of Msx1, Msx2 or Lmx1a, either individually or in combination, in stem cells.

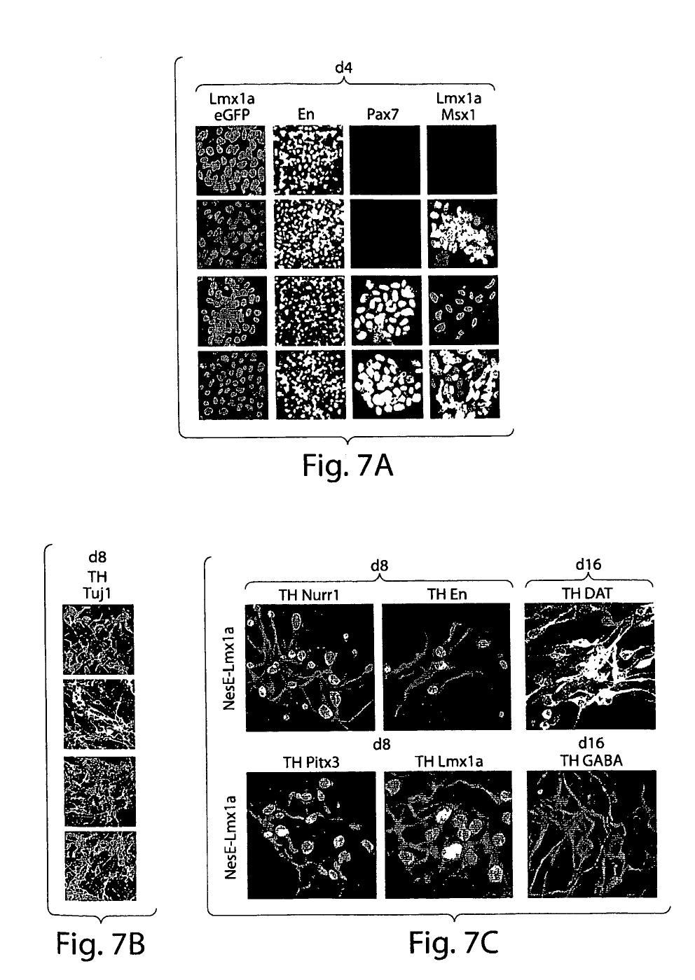

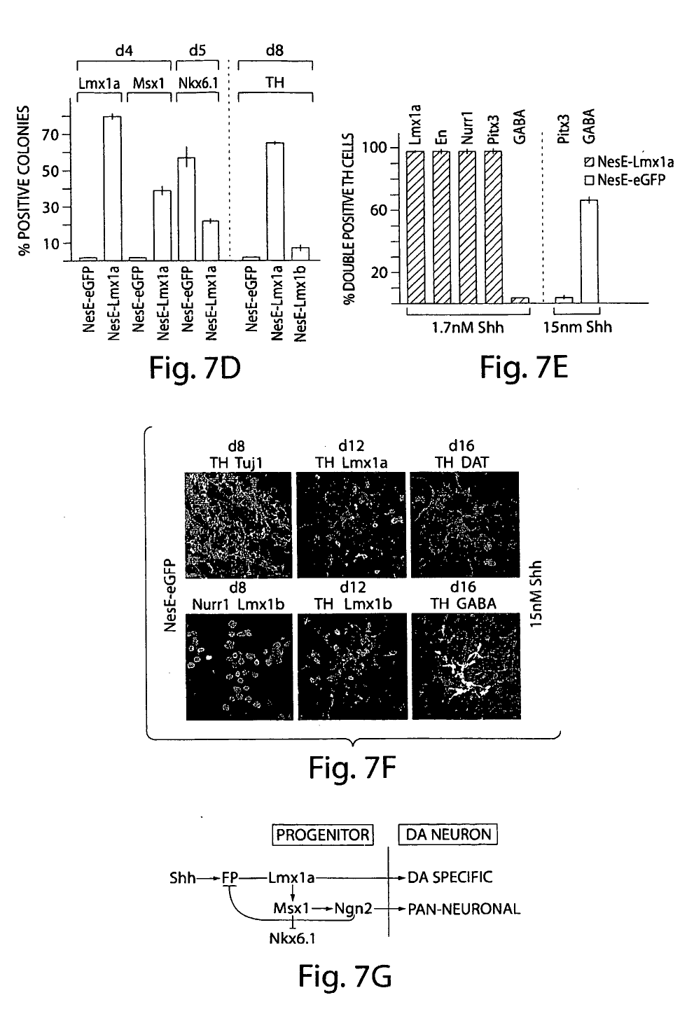

The protocol for DA neuron engineering described herein is uniquely efficient. First, in stably transformed ES cells the process of DA neuron differentiation is extremely robust. Almost all (80-100%) Lmx1a-ES and approximately 50% of Msx1-ES cells cells differentiate into DA neurons. Second, experiments clearly verify that the differentiated DA neurons are authentic as they express all analyzed markers that are expected to be expressed in bona fide DA neurons (see FIG. 7). Applicants are not aware of any other protocol that generates verified authentic DA neurons. Third, after transplantation into rat brains, these cells are integrated in a way that mimics primary DA neurons.

The Msx1 gene (msh homeo box homolog 1; NM_{—}002448, UniGene Hs.424414, corresponding murine homolog; NM_{—}010835) is deleted in patients with Wolf-Hirschhorn syndrome. This gene is also called HOX7, HYDI and OFC5. The Msx2 gene (msh homeo box homolog 2; NM_{—}002449, UniGene Hs.89404), when mutated, is associated with craniosynostosis and enlarged parietal foramina. This gene is also called FPP, MSH, PFM, CRS2, HOX8 and PFM1. The murine Msx3 gene (msh homeo box homolog 3; NM_{—}010836, UniGene Mm.4816) is highly homologous to the Msx1 and Msx2 genes.

The Lmx1a gene (LIM homeobox transcription factor 1 alpha; AH011517, NM_{—}177398, UniGene Hs.458270) is required for development of the roof plate (Chizhikov and Millen, Development. (2004) 131(11):2693-705). This gene is also called LMX1, LMX-1 and LMX1.1. The highly homologous gene Lmx1b (LIM homeobox transcription factor 1 alpha; NM_{—}002316, Hs.133709) is required for the normal development of dorsal limb structures, the glomerular basement membrane, the anterior segment of the eye, and dopaminergic and serotonergic neurons.

The sequence encoding Msx1 preferably is SEQ ID NO:1 or the coding region thereof, the sequence encoding Msx2 preferably is SEQ ID NO:3 or the coding region thereof, the sequence encoding Msx3 preferably is SEQ ID NO:5 or the coding region thereof, the sequence encoding Lmx1a preferably is SEQ ID NO:7 or the coding region thereof, and the sequence encoding Lmx1b preferably is SEQ ID NO:9 or the coding region thereof.

Orthologs (of non-human species; i.e., homologs encoded by different genomes) of the dopamine neuron determinants also are expected to promote DA neuron differentiation. The high evolutionary conservation of these genes suggests that the orthologs from any vertebrate species are likely have the potential to promote DA neuron differentiation from any vertebrate stem cell. Modified forms and derivatives of the dopamine neuron determinants also are expected to promote DA neuron differentiation. For example, a protein containing the DNA binding domain from Lmx1a and a functional domain from another transcription factor is expected to have similar gene regulatory functions as wild-type Lmx1a. Such orthologs, modified forms and derivatives can be identified, isolated and used in accordance with the invention using standard molecular biology techniques. The differentiation potential of any given ortholog, modified form or derivative can be tested readily using the methods described herein.

The invention thus provides cells that over-express one of more of the dopamine neuron determinants Msx1, Msx2, Msx3, Lmx1a and Lmx1b. In particular, the invention is provides cells that can differentiate to dopamine neurons, e.g., stem cells, neural precursors, neural progenitors, neuronal precursors and neuronal progenitors, as well as dopamine neurons that over-express the dopamine neuron determinant(s) or that have been differentiated from stem cells, precursors or progenitors by over-expression of dopamine neuron determinants. In addition, the invention provides methods that use any cell that can be made to differentiate into a neuron. For example, it is possible in theory that any cell, e.g. liver cells or fibroblasts, can undergo such transformations under appropriate conditions. Thus, the invention includes non-neural, neural and/or neuronal progenitors or precursors in which one or more dopamine neuron determinants is over-expressed. The invention also includes the use of such cells.

As used herein, “stem cells” are undifferentiated cells that have the potential to produce many or all kinds of cells in the body, including, in particular, neurons. Stem cells include embryonic stem cells and adult stem cells derived from or obtained from a variety of tissues, including skin, umbilical cord blood, hair follicles, muscle, bone marrow, liver, fat, blood, bone, kidney, gut, prostate and bladder, etc. Embryonic stem (ES) cells are clonal cell lines derived from the inner cell mass of developing blastocysts. ES cells can renew themselves and are pluripotent, i.e., can differentiate into a broad spectrum of derivatives of the three embryonic germ layers: ectoderm, mesoderm, and endoderm. Adult stem cells are undifferentiated cells found in a differentiated tissue that can renew itself and (with certain limitations) differentiate to yield all the specialized cell types of the tissue from which it originated. Under certain conditions, adult stem cells from one tissue can give rise to cell types of a completely different tissue, a phenomenon known as plasticity. As used herein, “neural precursors” and “neural progenitors” include those cells that are still uncommitted to neuronal vs. astroglial or oligodendroglial fate. As used herein, “neuronal precursors” and “neuronal progenitors” are already committed to become neurons of some type. The set of neural precursors and neural progenitors includes neuronal precursors and neuronal progenitors. As used herein, “non-neural precursors” and “non-neural progenitors” are already committed to become a cell type other than a neuron.

As used herein, “over-expression” includes increased expression of the dopamine neuron determinant(s) by adding to the cells exogenous nucleic acids that encode dopamine neuron determinant(s) (e.g., expression vectors). Over-expression also includes expressing the dopamine neuron determinant(s) by other means, including gene activation technologies such as turning on/increasing expression of endogenous dopamine neuron determinant gene(s) by activation of the endogenous promoter, and homologous recombination of the dopamine neuron determinant gene(s) to introduce a different promoter/enhancer to regulate endogenous gene. Various methods for increasing expression of genes are known to those of skill in the art and are considered useful for carrying out the invention. In certain embodiments (e.g., in vivo differentiation), the expression of the dopamine neuron determinant(s) is “ectopic”, which refers to forced expression of genes at sites where the gene is not normally expressed in vivo.

Preferably Msx1, Msx2, Msx3, Lmx1a and/or Lmx1b are expressed recombinantly in the cells. As used herein, “recombinant expression” means expression directed by a nucleic acid that has been produced by genetic engineering. Typically, this means that the recombinant expression is effectuated by operably linking the Msx1, Msx2, Msx3, Lmx1a and/or Lmx1b genes to an exogenous promoter and/or enhancer sequence, for example in an expression vector. The recombinant expression vector is introduced into the cells to increase expression of the dopamine neuron determinants. Introduction of expression vectors is well known in the art, and includes transfection, infection, recombination, electroporation, microinjection etc., as is described in further detail below.

Certain promoter sequences and/or enhancer sequences are preferred for inclusion in expression vectors to drive expression of the dopamine neuron determinants in a manner and amount that is effective to induce differentiation of the cells to DA neurons. Preferred promoters include the promoter from the herpes simplex virus thymidine kinase (tk) gene, nestin promoter and the Rous sarcoma virus LTR promoter (e.g., as present in RCAS vectors). Preferred enhancers include the Sonic hedgehog (Shh) enhancer and the nestin enhancer. Various combinations of promoters and enhancers can be prepared and are considered useful for the invention. For example, in the Nestin-expression vectors described in the Examples, the tk promoter is linked to a nestin enhancer.

In each of the embodiments of the invention, the preferred cell type is human. Thus human stem cells, neural precursors and progenitors, neuronal precursors and progenitors, and neurons are preferred.

To increase expression of one or more dopamine neuron determinant gene products (i.e., nucleic acids and/or polypeptides), a dopamine neuron determinant nucleic acid, in one embodiment, is operably linked to a gene expression sequence which directs the expression of the dopamine neuron determinant nucleic acid within a eukaryotic or prokaryotic cell.

The “gene expression sequence” is any regulatory nucleotide sequence, such as a promoter sequence, enhancer sequence or promoter-enhancer combination, which facilitates the efficient transcription and/or translation of the dopamine neuron determinant nucleic acid to which it is operably linked. The gene expression sequence may, for example, be a mammalian or viral promoter, such as a constitutive or inducible promoter. Constitutive mammalian promoters include, but are not limited to, the promoters for the following genes: hypoxanthine phosphoribosyl transferase (HPRT), adenosine deaminase, pyruvate kinase, β-actin promoter and other constitutive promoters. Exemplary viral promoters which function constitutively in eukaryotic cells include, for example, promoters from the simian virus, papilloma virus, adenovirus, human immunodeficiency virus (HIV), Rous sarcoma virus, cytomegalovirus, the long terminal repeats (LTR) of Rous sarcoma virus and other retroviruses, and the thymidine kinase promoter of herpes simplex virus. Other constitutive promoters are known to those of ordinary skill in the art. The promoters useful as gene expression sequences of the invention also include inducible promoters. Inducible promoters are expressed in the presence of an inducing agent. For example, the metallothionein promoter is induced to promote transcription and translation in the presence of certain metal ions. Other inducible promoters are known to those of ordinary skill in the art.

Preferably, however, the promoter is a promoter that directs gene expression in a cell-type-specific and developmental stage-specific manner, i.e., specific for dopamine neurons and/or precursors or progenitors thereof. The promoters for the dopamine neuron determinant genes identified herein are examples of such cell-type and developmental-stage specific promoters.

In general, the gene expression sequence shall include, as necessary, 5′ non-transcribed and 5′ non-translated sequences involved with the initiation of transcription and translation, respectively, such as a TATA box, capping sequence, CAAT sequence, and the like. Especially, such 5′ non-transcribed sequences will include a promoter region which includes a promoter sequence for transcriptional control of the operably joined dopamine neuron determinant nucleic acid. The gene expression sequences optionally includes enhancer sequences or upstream activator sequences as desired. The dopamine neuron determinant nucleic acids also can be linked to 3′ non-transcribed and 3′ non-translated sequences, which may be useful for termination of transcription and/or translation, enhancing transcription or translation (e.g., downstream enhancers), etc.

A dopamine neuron determinant nucleic acid sequence and the gene expression sequence are said to be “operably linked” when they are covalently linked in such a way as to place the transcription and/or translation of the dopamine neuron determinant coding sequence under the influence or control of the gene expression sequence. If it is desired that the dopamine neuron determinant sequence be translated into a functional protein, two DNA sequences are said to be operably linked if induction of a promoter in the 5′ gene expression sequence results in the transcription of the dopamine neuron determinant sequence and if the nature of the linkage between the two DNA sequences does not (1) result in the introduction of a frame-shift mutation, (2) interfere with the ability of the promoter region to direct the transcription of the dopamine neuron determinant sequence, or (3) interfere with the ability of the corresponding RNA transcript to be translated into a protein. Thus, a gene expression sequence would be operably linked to a dopamine neuron determinant nucleic acid sequence if the gene expression sequence were capable of effecting transcription of that dopamine neuron determinant nucleic acid sequence such that the resulting transcript can be translated into the desired protein or polypeptide.

The dopamine neuron determinants of the invention can be delivered to the eukaryotic or prokaryotic cell alone or in association with a vector. In its broadest sense, a “vector” is any vehicle capable of facilitating: (1) delivery of a dopamine neuron determinant nucleic acid or polypeptide to a target cell or (2) uptake of a dopamine neuron determinants nucleic acid or polypeptide by a target cell. Preferably, the vectors transport the dopamine neuron determinant nucleic acid or polypeptide into the target cell with reduced degradation relative to the extent of degradation that would result in the absence of the vector. Optionally, a “targeting ligand” can be attached to or incorporated in the vector to selectively deliver the vector to a cell which expresses on its surface the cognate receptor (e.g. a receptor, an antigen recognized by an antibody) for the targeting ligand. In this manner, the vector (containing a dopamine neuron determinant nucleic acid or polypeptide) can be selectively delivered to a specific cell. In general, the vectors useful in the invention are divided into two classes: biological vectors and chemical/physical vectors. Biological vectors are more useful for delivery/uptake of dopamine neuron determinant nucleic acids to/by a target cell. Chemical/physical vectors are more useful for delivery/uptake of dopamine neuron determinant proteins to/by a target cell.

Biological vectors include, but are not limited to, plasmids, phagemids, viruses, other vehicles derived from viral or bacterial sources that have been manipulated by the insertion or incorporation of the nucleic acid sequences of the invention, and free nucleic acid fragments which can be attached to the nucleic acid sequences of the invention. Viral vectors are a preferred type of biological vector and include, but are not limited to, nucleic acid sequences from the following viruses: retroviruses, such as Moloney murine leukemia virus; Harvey murine sarcoma virus; murine mammary tumor virus; Rous sarcoma virus (including the RCAS vector used in the Examples); adenovirus; adeno-associated virus; SV40-type viruses; polyoma viruses; Epstein-Barr viruses; papilloma viruses; herpes virus; vaccinia virus; and polio virus. One can readily employ other vectors not named but known in the art.

Preferred viral vectors are based on non-cytopathic eukaryotic viruses in which non-essential genes have been replaced with the gene of interest. Non-pathogenic and non-cytopathic neurotropic virus vectors are preferred, which can be weakened forms of pathogenic neurotropic viruses. Non-cytopathic viruses include retroviruses, the life cycle of which involves reverse transcription of genomic viral RNA into DNA with subsequent proviral integration into host cellular DNA. In general, the retroviruses are replication-deficient (i.e., capable of directing synthesis of the desired proteins, but incapable of manufacturing an infectious particle). Such genetically altered retroviral expression vectors have general utility for the high-efficiency transduction of genes in vivo. Standard protocols for producing replication-deficient retroviruses (including the steps of incorporation of exogenous genetic material into a plasmid, transfection of a packaging cell line with plasmid, production of recombinant retroviruses by the packaging cell line, collection of viral particles from tissue culture media, and infection of the target cells with viral particles) are well known in the art, and thus not detailed here.

Another preferred virus for certain applications is the adeno-associated virus, a double-stranded DNA virus. The adeno-associated virus can be engineered to be replication-deficient and is capable of infecting a wide range of cell types and species. It further has advantages, such as heat and lipid solvent stability; high transduction frequencies in cells of diverse lineages, including hematopoietic cells; and lack of superinfection inhibition thus allowing multiple series of transductions. Reportedly, the adeno-associated virus can integrate into human cellular DNA in a site-specific manner, thereby minimizing the possibility of insertional mutagenesis and variability of inserted gene expression. In addition, wild-type adeno-associated virus infections have been followed in tissue culture for greater than 100 passages in the absence of selective pressure, implying that the adeno-associated virus genomic integration is a relatively stable event. The adeno-associated virus can also function in an extrachromosomal fashion.

Expression vectors containing all the necessary elements for expression of dopamine neuron determinant genes are commercially available and known to those skilled in the art. See, e.g., Sambrook et al., Molecular Cloning: A Laboratory Manual, Second Edition, Cold Spring Harbor Laboratory Press, 1989. Cells are genetically engineered by the introduction into the cells of heterologous DNA (RNA) encoding a dopamine neuron determinant polypeptide or fragment or variant thereof. That heterologous DNA (RNA) is placed under operable control of transcriptional elements to permit the expression of the heterologous DNA in the host cell.

Preferred systems for mRNA expression in mammalian cells include those described herein, and others such as the pcDNA series of vectors (available from Invitrogen, Carlsbad, Calif.) that contain a selectable marker such as a gene that confers G418 resistance (which facilitates the selection of stably transfected cell lines) and the human cytomegalovirus (CMV) enhancer-promoter sequences. Additionally, suitable for expression in primate or canine cell lines is the pCEP4 vector (Invitrogen), which contains an Epstein Barr virus (EBV) origin of replication, facilitating the maintenance of plasmid as a multicopy extrachromosomal element.

In addition to the biological vectors, chemical/physical vectors may be used to deliver a dopamine neuron determinant nucleic acid or polypeptide to a target cell and facilitate uptake thereby. As used herein, a “chemical/physical vector” refers to a natural or synthetic molecule, other than those derived from bacteriological or viral sources, capable of delivering the isolated dopamine neuron determinant nucleic acid or polypeptide to a cell.

A preferred chemical/physical vector of the invention is a colloidal dispersion system. Colloidal dispersion systems include lipid-based systems including oil-in-water emulsions, micelles, mixed micelles, and liposomes. A preferred colloidal system of the invention is a liposome. Liposomes are artificial membrane vesicles which are useful as a delivery vector in vivo or in vitro. It has been shown that large unilamellar vesicles (LUV), which range in size from 0.2-4.0 μm can encapsulate large macromolecules. RNA, DNA, and intact virions can be encapsulated within the aqueous interior and be delivered to cells in a biologically active form. In order for a liposome to be an efficient nucleic acid transfer vector, one or more of the following characteristics should be present: (1) encapsulation of the nucleic acid of interest at high efficiency with retention of biological activity; (2) preferential and substantial binding to a target cell in comparison to non-target cells; (3) delivery of the aqueous contents of the vesicle to the target cell cytoplasm at high efficiency; and (4) accurate and effective expression of genetic information.

Liposomes may be targeted to a particular tissue by coupling the liposome to a specific ligand such as a monoclonal antibody, sugar, glycolipid, or protein. Ligands which may be useful for targeting a liposome to a particular cell will depend on the particular cell or tissue type. Additionally when the vector encapsulates a nucleic acid, the vector may be coupled to a nuclear targeting peptide, which will direct the dopamine neuron determinant nucleic acid to the nucleus of the host cell.

Liposomes are commercially available from a variety of vendors, for example, LIPOFECTIN® and LIPOFECTAMINE™ (Invitrogen), which are formed of cationic lipids such as N-[1-(2, 3 dioleyloxy)-propyl]-N,N,N-trimethylammonium chloride (DOTMA) and dimethyl dioctadecylammonium bromide (DDAB). Methods for making liposomes are well known in the art and have been described in many publications.

Other exemplary compositions that can be used to facilitate uptake by a target cell of the dopamine neuron determinant nucleic acids include calcium phosphate and other chemical mediators of intracellular transport, microinjection compositions, electroporation and homologous recombination compositions (e.g., for integrating a dopamine neuron determinant nucleic acid into a preselected location within a target cell chromosome).

The invention also embraces so-called expression kits, which allow the artisan to prepare a desired expression vector or vectors. Such expression kits include at least separate portions of the previously discussed dopamine neuron determinant coding sequences. Other components may be added, as desired, as long as the previously mentioned sequences, which are required, are included.

Dopamine neuron determinant cDNA sequences can thus be used in expression vectors to transfect host cells and cell lines, whether prokaryotic (e.g., E. coli), or eukaryotic (e.g., neurons, stem cells, neural precursor, etc.). Especially useful are mammalian cells such as human, pig, goat, primate, mouse, rat, etc., which can be used for the identification of molecules that regulate the function of dopamine neuron determinants selectively or preferentially (e.g., by screening chemical compound libraries). The cells may be of a wide variety of tissue types, and include primary cells and cell lines. Specific examples include stem cells, neural precursors or progenitors, neuronal precursors or progenitors, neuronal cell lines including PC12 cells, and Xenopus oocytes.

The expression vectors can be used in the various therapeutic, diagnostic and screening methods described herein. For example, expression of dopamine neuron determinant gene products may be performed to obtain polypeptide for antibodies or other diagnostic and therapeutic reagents. Expression of dopamine neuron determinant gene products may be used in therapies for neurodegenerative disease and other disorders in which production of dopamine neuron determinants is desirable, e.g., by increasing expression of dopamine neuron determinant gene products in neurons in vitro for eventual transplantation or in vivo increase dopamine neuron determinants in situ.

Also provided are cell lines and cell populations of the cells that over-express the dopamine neuron determinant(s). The cells may be provided in a carrier, such as a pharmaceutically acceptable carrier for therapeutic purposes, or a cell freezing medium or cell storage medium. Cultures of the cells, in culture plates, culture dishes, roller bottles, multiwell plates, etc. also are provided. The cultures are prepared and maintained in accordance with standard protocols that are well known in the art.

Importantly, the identification of dopamine neuron determinants permits directed differentiation of various cells to dopamine (DA) neurons. Thus the invention provides methods for producing DA neurons, in which the expression of Msx1, Msx2, Msx3, Lmx1a and/or Lmx1b in a cell is increased. The cells are cultures under conditions and for a time sufficient to permit differentiation of the cell to a DA neuron. The dopamine neuron determinants are increased, for example, by over-expressing one or more nucleic acids that encode Msx1, Msx2, Msx3, Lmx1a and/or Lmx1b proteins, as described above and elsewhere herein. Culturing can be performed in vitro, ex vivo, or in vivo.

In one aspect of the foregoing methods, the invention provides methods for altering the phenotype of motor neuron progenitor cells, e.g., by respecifying differentiation of the motor neuron progenitor cells. When the expression of Msx1, Msx2, Msx3, Lmx1a and/or Lmx1b is increased in motor neuron progenitor cells (i.e., as described above for differentiation of cells to DA neurons), the cells do not differentiate to motor neurons, but rather are directed to differentiate into dopamine neurons or DA neuron progenitor cells.

The dopamine neurons of the invention (e.g., those produced by methods of the invention) can be transplanted into subjects in need of such treatment. Thus, cell transplantation methods are provided in accordance with the invention, which are useful for the treatment of diseases and disorders in which dopamine neurons have degenerated. Diseases treatable by transplantation of dopamine neurons include Parkinson's disease and other neurodegenerative motor disorders. For treatment of subjects having or suspected of having Parkinson's disease, DA neurons or precursors or progenitors with increased expression of dopamine neuron determinants are obtained and transplanted, preferably into the brain, e.g. the striatum, of the subject. The DA neurons preferably are those that over-express Msx1, Msx2, Msx3, Lmx1a and/or Lmx1b, although DA neurons produced by increasing the expression of Msx1, Msx2, Msx3, Lmx1a and/or Lmx1b by other means, also are useful in these methods.

As used herein, a “subject” is any organism having dopamine neurons, preferably a mammal, more preferably a primate, and most preferably a human. Additional subjects include house pets (e.g., dogs, cats, fish, etc.), agricultural stock animals (e.g., cows, horses, pigs, chickens, etc.), laboratory animals (e.g., mice, rats, rabbits, etc.), zoo animals (e.g., lions, giraffes, etc.), but are not so limited. Preferred subjects are human subjects.

The dopamine neuron determinants provided herein also can be used to promote growth of dopamine neurons in situ or in culture. These methods can be carried out by modulating the expression or activity of one or more dopamine neuron determinant gene products in stem cells and neural and/or neuronal progenitors or precursors. Cells treated in accordance with this aspect of the invention can be used in cell transplantation, e.g., for treatment purposes. DA neurons (and stem cells, precursors and progenitors capable of differentiating into DA neurons) also can be treated in situ using activators or expression modulators of dopamine neuron determinants. In certain embodiments, expression is increased by expressing exogenous nucleic acid molecules that encode one or more dopamine neuron determinant gene products in the population of stem cells, neural and/or neuronal progenitors or precursors, preferably using an expression vector. In other embodiments, a population of stem cells, neural and/or neuronal progenitors or precursors is contacted with a pharmacological molecule that induces increased expression of the one or more gene products.

After differentiating stem cells or neural or neuronal precursors or progenitors to DA neurons, the DA neurons can optionally be cultured to expand the population of cells (e.g., for cell transplantation), to subject the cells to further differentiation, to use the cells in screening assays, etc. For cell transplantation, an expanded DA neuron population or progeny cells produced therefrom are administered in an effective amount to a patient. Preferred patients are those that have or are suspected of having a neurodegenerative disease or disorder, particularly Parkinson's disease or other disease or disorder in which there is degeneration of DA neurons.

Potential targets for therapy in accordance with the invention include neurodegenerative disorders or diseases. A “neurodegenerative disease” or a “neurodegenerative disorder” is defined herein as a condition in which there is progressive loss of neurons in the nervous system. Most of the chronic neurodegenerative diseases are typified by onset during the middle adult years and lead to rapid degeneration of specific subsets of neurons within the nervous system, ultimately resulting in premature death. Other neurodegenerative disorders can result from exposure to neurotoxic chemicals, such as 1′-methyl-4-phenyl-1,2,3,6-tetrahydropyridine (MPTP) and its toxic metabolite 1-methyl-4-phenylpyridinium ion (MPP(+)), 3-nitropropionic acid (3NP), 6-hydroxydopamine (6-OHDA), the pesticides rotenone, deguelin, paraquat and diquat, and the fungicide maneb and its major active element, manganese ethylene-bis-dithiocarbamate (Mn-EBDC).

Parkinson's disease (paralysis agitans) is a common neurodegenerative disorder that appears in mid to late life. Familial and sporadic cases occur, although familial cases account for only 1-2 percent of the observed cases. Patients frequently have nerve cell loss with reactive gliosis and formation of Lewy bodies in the substantia nigra and locus coeruleus of the brainstem. Similar changes are observed in the nucleus basalis of Meynert and, in the long term, the nerve cell loss may be quite widespread. As a class, the nigrostriatal dopaminergic neurons seem to be most affected. The disorder generally develops asymmetrically with tremors in one hand or leg and progresses into symmetrical loss of voluntary movement. Eventually, the patient becomes incapacitated by rigidity and tremors. In the advanced stages the disease is frequently accompanied by dementia. Diagnosis of both familial and sporadic cases of Parkinson's disease can only be made after the onset of the disease.

Parkinsonism associated with multiple system atrophy (MSA-P) is an atypical Parkinsonism that was first identified by Adams and colleagues in four patients with severe, progressive Parkinsonism in whom postmortem examination disclosed extensive loss of striatal neurons, particularly in the putamen, as well as degeneration of the substantia nigra (Adams et al., J Neuropathol Exp Neurol 1964; 23:584-608), while Lewy bodies, the histopathological hallmark of idiopathic PD, were absent (Andrews et al., Arch Neurol 1970; 23:319-329). Clinically, MSA-P is an akinetic-rigid syndrome characterized by gait disturbance, rigidity, progressive bradykinaesia, and a poor response to dopaminergic therapy.

Sporadic and familiar progressive supranuclear palsy (PSP), first described in 1964, is an atypical Parkinsonism characterized by loss of balance, changes in personality, blurring of vision and problems controlling eye movement. PSP results from a gradual deterioration of neuron in several locations in the brainstem including, the substantia nigra.

Corticobasal degeneration (CBD) is another neurodegenerative disorder characterized by degeneration of dopamine neurons. The most characteristic initial motor symptoms are akinesia, rigidity, and apraxia. Dystonia and alien limb phenomena are frequently observed.

Assays can be performed to screen and/or determine whether a molecule has the ability to increase dopamine neuron determinant gene product activity, and whether the increase is selective or preferential. As used herein, “increasing dopamine neuron determinant gene product activity” refers to increasing by at least 10% dopamine neuron determinant gene product expression or activity, preferably increasing by at least 25%, and more preferably increasing by at least 40% as measured by any of the methods well known in the art or as provided herein. Exemplary assays of dopamine neuron determinant gene product expression include RT-PCR, microarray analysis, and ELISA and other antibody-based detection assays. Assays that measure dopamine production by cells also can be used, alone or in combination with the assays of dopamine neuron determinant expression. By “selective increase” is meant that the compound increases gene product expression or activity in a dopamine neuron determinant specific manner, e.g., resulting in differentiation of stem cells or neural or neuronal precursors or progenitors to DA neurons but not significant amounts of other types of neurons. By “preferential increase” is meant that the compound increases dopamine neuron determinant gene product expression or activity by at least about 5% more than other gene product expression or activity for differentiation to other neuron types. Preferably, the preferential increase is at least about 10% more for dopamine neuron determinants, more preferably at least about 20% more, still more preferably at least about 30% more, yet more preferably at least about 40%, and most preferably at least about 50% more for dopamine neuron determinants. Greater differences in increases of dopamine neuron determinant gene product expression or activity than that of non-dopamine neuron determinant gene products is contemplated, from 51% all the way up to about 99%, at which point the increase may be considered selective. Molecules may selectively or preferentially increase dopamine neuron determinant gene product expression or activity by modulating transcription, translation, or activity of the dopamine neuron determinant gene products.

It is also contemplated that one can screen for inhibitors of the expression or activity of dopamine neuron determinants in a similar manner as that described above for molecules that increase dopamine neuron determinant expression or activity. Inhibitors of the expression or activity of dopamine neuron determinants are useful for ensuring that stem cells or neural or neuronal precursors or progenitors do not differentiate into dopamine neurons, or do so at a lesser frequency. Thus, if one wanted to differentiate cells into non-dopamine neurons, the use of inhibitors of dopamine neuron determinants would aid in obtaining the desired neuron type rather than dopamine neurons.

In screening for modulators of dopamine neuron determinant gene products, including inhibitors and activators (i.e. antagonists and agonists), it is preferred that molecules (e.g., libraries of potential modulators) are tested for modulation of dopamine neuron determinant gene product expression at a variety of developmental stages in stem cells, neural precursors or progenitors, neuronal precursors or progenitors, and/or neurons. Such compounds are useful for selectively modulating dopamine neuron determinant gene products in the various stages of development, and may be used combinatorially and/or sequentially to direct DA neuron development (or to direct development of other neuron types in the case of dopamine neuron determinant inhibitors). For example, stem cells may be treated with one or more modulators in a sequential manner in order to mimic the natural development of dopamine neurons. Other uses will be apparent to one of ordinary skill in the art.

The invention further provides efficient methods of identifying compounds, pharmacological agents or lead compounds for agents useful in the treatment of conditions associated with neurodegeneration, particularly those conditions involving degeneration of DA neurons, and the compounds and agents so identified. Also included in the invention are screens for compounds that modulate (increase or decrease) behavior of DA neurons in other ways, e.g., their ability to synthesize, store and release dopamine and/or the growth and/or survival of the DA neurons. Such modulation may be important in neurodegenerative diseases such as Parkinson's disease, but may also be important in other diseases or disorders, e.g. schizophrenia.

In some embodiments of the invention, increasing DA neuron survival under toxic stress is the end point analyzed. One method to induce stress is to contact DA neurons with toxic compounds, such as those described above, including 6-hydroxy-dopamine or 1-methyl-4-phenyl-pyridinium (MPP(+)). Alternatively, ES cells can be genetically engineered in ways that will induce pathology, e.g., by expressing the protein alpha-synuclein. This protein has been implicated in the normal process of Parkinson's disease pathology and is known to cause cell death when overexpressed in neurons.

Also provided by the invention are methods for identifying compounds, pharmacological agents or lead compounds for agents useful in the differentiation of stem cells, neural and/or neuronal progenitors or precursors to dopamine (DA) neurons. In these methods, cells are contacted with one or more molecules, and the effects of the molecules on the expression of dopamine neuron determinants are then determined. Compounds that increase expression of the DA neuron determinants Msx1, Msx2, Msx3, Lmx1a and/or Lmx1b are compounds that can be used for differentiation of stem cells, neural and/or neuronal progenitors or precursors to DA neurons.

Generally, the screening methods involve assaying for compounds which increase the expression or activity of dopamine neuron determinant gene products. Such methods are adaptable to automated, high throughput screening of compounds. Examples of such methods are described in U.S. Pat. No. 5,429,921.

One common set of compounds for screening in the identification methods of the invention is a library of molecules. Libraries are groups of compounds of similar or divergent structures and sources. For example, some libraries of compounds include natural products, such as compounds produced by microorganisms like bacteria or fungi. Such libraries may be of a common source, but of highly divergent structures. Another example is libraries generated by combinatorial chemistry. Molecules in combinatorial libraries typically have a common core structure, with varying amounts of additional groups and kinds of groups added to the core structure to form a set of compounds that shares some degree of structural similarity. In combinatorial libraries based on a known molecular structure, e.g., synthesized to identify analogs of the known molecule, the degree of structural similarity typically will be higher than in other combinatorial libraries. Still another example is a library of known drug molecules; molecules in these libraries tend to have little structural relatedness, and are typically of widely divergent sources (including natural products and synthetic molecules).

A variety of assays for pharmacological agents are provided, including, labeled in vitro protein binding assays, gene expression assays, etc. For example, protein binding screens are used to rapidly examine the binding of candidate compounds to a dopamine neuron determinant polypeptide. Gene expression screens examine the modulation of dopamine neuron determinant gene product expression via methods such as those detailed in the Examples. The candidate compounds can be derived from, for example, combinatorial peptide libraries, combinatorial chemical compound libraries, and natural products libraries. Convenient reagents for such assays are known in the art.

For certain methods, such as dopamine neuron determinant gene expression assays, cells that express a determinable quantity of dopamine neuron determinant gene products are used; the effect of the test molecules on dopamine neuron determinant gene product expression is determined. For example, cells that express a baseline amount of dopamine neuron determinant gene products (i.e., no expression or more) under assay conditions are contacted with molecules to determine the effect of the molecules on the expression of the dopamine neuron determinant gene products. Molecules that increase expression of dopamine neuron determinant gene products are identified as those that increase the amount of expression of dopamine neuron determinant gene products above the baseline amount.

For other methods, dopamine neuron determinant gene products can be added to an assay mixture as an isolated polypeptide (where binding of a candidate pharmaceutical agent is to be measured) or as a cell or other membrane-encapsulated space which includes a dopamine neuron determinant polypeptide. In the latter assay configuration, the cell or other membrane-encapsulated space can contain the dopamine neuron determinant gene product as a preloaded polypeptide or as a nucleic acid (e.g., a cell transfected with an expression vector containing a nucleic acid that encodes a dopamine neuron determinant polypeptide). In the assays described herein, the dopamine neuron determinant polypeptide can be produced recombinantly, or isolated from biological extracts, but preferably is synthesized in vitro. Dopamine neuron determinant polypeptides encompass chimeric proteins comprising a fusion of a dopamine neuron determinant polypeptide with another polypeptide, e.g., a polypeptide capable of providing or enhancing protein-protein binding, or enhancing stability of the dopamine neuron determinant polypeptide under assay conditions. A polypeptide fused to a dopamine neuron determinant polypeptide or fragment thereof may also provide means of readily detecting the fusion protein, e.g., by immunological recognition or by fluorescent labeling.

For the cell-based assays described herein, preferred cell types are stem cells (particularly ES cells), neural precursors or progenitors, neuronal precursors or progenitors, and neurons. Matched control cells can be used in the assays, e.g., cells that do not express dopamine neuron determinant gene products.

The assay mixture also comprises a candidate compound molecule. Typically, a plurality of assay mixtures are run in parallel with different compound concentrations to obtain a different response to the various concentrations. Typically, one of these concentrations serves as a negative control, i.e., at zero concentration of compound or at a concentration of compound below the limits of assay detection. Candidate compounds encompass numerous chemical classes, although typically they are organic compounds. Preferably, the candidate compounds are small organic compounds, i.e., those having a molecular weight of more than 50 yet less than about 2500.

Candidate compounds comprise functional chemical groups necessary for structural interactions with polypeptides, and typically include at least an amine, carbonyl, hydroxyl or carboxyl group, preferably at least two of the functional chemical groups and more preferably at least three of the functional chemical groups. The candidate compounds can comprise cyclic carbon or heterocyclic structure and/or aromatic or polyaromatic structures substituted with one or more of the above-identified functional groups. Candidate compounds also can be biomolecules such as peptides, saccharides, fatty acids, sterols, isoprenoids, purines, pyrimidines, derivatives or structural analogs of the above, or combinations thereof and the like. Where the compound is a nucleic acid, the compound typically is a DNA or RNA molecule, although modified nucleic acids having non-natural bonds or subunits are also contemplated. Thus, antisense and siRNA molecules can be tested for inhibition of dopamine neuron determinant gene product expression by these assays and other standard assays of nucleic acid expression, such as microarrays and PCR. Utilizing the cell-based assays described above allows the identification of antisense and siRNA molecules that inhibit function of dopamine neuron determinant gene products.

Candidate compounds are obtained from a wide variety of sources including libraries of synthetic or natural compounds. For example, numerous means are available for random and directed synthesis of a wide variety of organic compounds and biomolecules, including expression of randomized oligonucleotides, synthetic organic combinatorial libraries, phage display libraries of random peptides, and the like. Alternatively, libraries of natural compounds in the form of bacterial, fungal, plant and animal extracts are available or readily produced. Additionally, natural and synthetically produced libraries and compounds can be readily modified through conventional chemical, physical, and biochemical means. Further, known pharmacological compounds may be subjected to directed or random chemical modifications such as acylation, alkylation, esterification, amidification, etc. to produce structural analogs of the compounds.

Candidate compounds can be selected randomly or can be based on existing compounds which bind to and/or modulate the function of dopamine neuron determinant gene products, e.g., following identification through screening. The structure of a candidate compound can be changed at one or more positions of the molecule to contain more or fewer chemical moieties or different chemical moieties. The structural changes made to the molecules in creating the libraries of analog compounds can be directed, random, or a combination of both directed and random substitutions and/or additions. One of ordinary skill in the art in the preparation of combinatorial libraries can readily prepare such libraries.

A variety of other reagents also can be included in the mixture. These include reagents such as salts, buffers, neutral proteins (e.g., albumin), detergents, etc. which may be used to facilitate optimal protein-protein and/or protein-nucleic acid binding. Such a reagent may also reduce non-specific or background interactions of the reaction components. Other reagents that improve the efficiency of the assay such as protease inhibitors, nuclease inhibitors, antimicrobial agents, and the like may also be used.

The mixture of the foregoing assay materials is incubated under conditions whereby, but for the presence of the candidate compound, a control amount of dopamine neuron determinant gene product expression or activity is obtained. For determining the binding of a candidate compound to a dopamine neuron determinant gene product, the mixture is incubated under conditions which permit binding. The order of addition of components, incubation temperature, time of incubation, and other parameters of the assay may be readily determined. Such experimentation merely involves optimization of the assay parameters, not the fundamental composition of the assay. Incubation temperatures typically are between 4° C. and 40° C. Incubation times preferably are minimized to facilitate rapid, high throughput screening, and typically are between 1 minute and 10 hours.

After incubation, the level of dopamine neuron determinant gene product expression or activity is detected by any convenient method available to the user. For cell free binding type assays, a separation step is often used to separate bound from unbound components. The separation step may be accomplished in a variety of ways. Conveniently, at least one of the components is immobilized on a solid substrate, from which the unbound components may be easily separated. The solid substrate can be made of a wide variety of materials and in a wide variety of shapes, e.g., microtiter plate, microbead, dipstick, resin particle, etc. The substrate preferably is chosen to maximize signal to noise ratios, primarily to minimize background binding, as well as for ease of separation and cost.

Separation may be effected for example, by removing a bead or dipstick from a reservoir, emptying or diluting a reservoir such as a microtiter plate well, rinsing a bead, particle, chromatographic column or filter with a wash solution or solvent. The separation step preferably includes multiple rinses or washes. For example, when the solid substrate is a microtiter plate, the wells may be washed several times with a washing solution, which typically includes those components of the incubation mixture that do not participate in specific bindings such as salts, buffer, detergent, non-specific protein, etc. Where the solid substrate is a magnetic bead, the beads may be washed one or more times with a washing solution and isolated using a magnet.

Detection may be effected in any convenient way for cell-based assays such as a gene expression assay as described herein. For cell free binding assays, one of the components usually comprises, or is coupled to, a detectable label. A wide variety of labels can be used, such as those that provide direct detection (e.g., radioactivity, luminescence, optical or electron density, etc). or indirect detection (e.g., epitope tag such as the FLAG epitope, enzyme tag such as horseradish peroxidase, etc.). The label may be bound to a dopamine neuron determinant polypeptide or the candidate compound.

A variety of methods may be used to detect the label, depending on the nature of the label and other assay components. For example, the label may be detected while bound to the solid substrate or subsequent to separation from the solid substrate. Labels may be directly detected through optical or electron density, radioactive emissions, nonradiative energy transfers, etc. or indirectly detected with antibody conjugates, streptavidin-biotin conjugates, etc. Methods for detecting the labels are well known in the art.

In another embodiment, the invention provides similar assays using dopamine neuron determinant gene products to identify modulators of dopamine neuron determinant gene product expression and function. In one particular embodiment, the modulator is an antisense oligonucleotide or siRNA molecule that selectively binds to a nucleic acid molecule, to reduce the expression of the encoded gene product in a cell. An example of this is the use of the antisense oligonucleotide or siRNA molecule to reduce the expression of dopamine neuron determinant genes to exclude or reduce differentiation into dopamine neurons. Another example is the use of antisense oligonucleotides or siRNA molecules to reduce the expression of dopamine neuron determinant genes at a particular stage of differentiation. Still another example is the use of the antisense oligonucleotides or siRNA molecules to determine whether the expression of a particular dopamine neuron determinant gene is essential to the dopamine neuron phenotype.

As used herein, the term “antisense oligonucleotide” or “antisense” describes an oligonucleotide that is an oligoribonucleotide, oligodeoxyribonucleotide, modified oligoribonucleotide, or modified oligodeoxyribonucleotide which hybridizes under physiological conditions to DNA comprising a particular gene or to an mRNA transcript of that gene and, thereby, inhibits the transcription of that gene and/or the translation of that mRNA. The antisense molecules are designed so as to interfere with transcription or translation of a target gene upon hybridization with the target gene or transcript. Those skilled in the art will recognize that the exact length of the antisense oligonucleotide and its degree of complementarity with its target will depend upon the specific target selected, including the sequence of the target and the particular bases which comprise that sequence.

As used herein, a “siRNA molecule” is a double stranded RNA molecule (dsRNA) consisting of a sense and an antisense strand, which are complementary (Tuschl, T. et al., 1999, Genes & Dev., 13:3191-3197; Elbashir, S. M. et al., 2001, EMBO J., 20:6877-6888). In one embodiment the last nucleotide at the 3′ end of the antisense strand may be any nucleotide and is not required to be complementary to the region of the target gene. The siRNA molecule may be 19-23 nucleotides in length in some embodiments. In other embodiments, the siRNA is longer but forms a hairpin structure of 19-23 nucleotides in length. In still other embodiments, the siRNA is formed in the cell by digestion of double stranded RNA molecule that is longer than 19-23 nucleotides. The siRNA molecule preferably includes an overhang on one or both ends, preferably a 3′ overhang, and more preferably a two nucleotide 3′ overhang on the sense strand. In another preferred embodiment, the two nucleotide overhang is thymidine-thymidine (TT). The siRNA molecule corresponds to at least a portion of the gene product of interest. In a preferred embodiment the first nucleotide of the siRNA molecule is a purine. Many variations of siRNA and other double stranded RNA molecules useful for RNAi inhibition of gene expression will be known to one of ordinary skill in the art.

The siRNA molecules can be plasmid-based. In a preferred method, a polypeptide encoding sequence of the gene of interest is amplified using the well known technique of polymerase chain reaction (PCR). The use of the entire polypeptide encoding sequence is not necessary; as is well known in the art, a portion of the polypeptide encoding sequence is sufficient for RNA interference. For example, the PCR fragment can be inserted into a vector using routine techniques well known to those of skill in the art. The insert can be placed between two promoters oriented in opposite directions, such that two complementary RNA molecules are produced that hybridize to form the siRNA molecule. Alternatively, the siRNA molecule is synthesized as a single RNA molecule that self-hybridizes to form a siRNA duplex, preferably with a non-hybridizing sequence that forms a “loop” between the hybridizing sequences. Preferably the nucleotide encoding sequence is part of the coding sequence of the gene of interest. The siRNA can be expressed from a vector introduced into cells.

Vectors comprising any of the nucleotide coding sequences of the invention are provided for production of siRNA, preferably vectors that include promoters active in mammalian cells. Non-limiting examples of vectors are the pSUPER RNAi series of vectors (Brummelkamp, T. R. et al., 2002, Science, 296:550-553; available commercially from OligoEngine, Inc., Seattle, Wash.). In one embodiment a partially self-complementary nucleotide coding sequence can be inserted into the mammalian vector using restriction sites, creating a stem-loop structure. In a preferred embodiment, the mammalian vector comprises the polymerase-III H1-RNA gene promoter. The polymerase-III H1-RNA promoter produces a RNA transcript lacking a polyadenosine tail and has a well-defined start of transcription and a termination signal consisting of five thymidines (T5) in a row. The cleavage of the transcript at the termination site occurs after the second uridine and yields a transcript resembling the ends of synthetic siRNAs containing two 3′ overhanging T or U nucleotides. Other promoters useful in siRNA vectors will be known to one of ordinary skill in the art.

Vector systems for siRNA expression in mammalian cells include pSUPER RNAi system described above. Other examples include but are not limited to pSUPER.neo, pSUPER.neo+gfp and pSUPER.puro (OligoEngine, Inc.); BLOCK-iT T7-TOPO linker, pcDNA1.2/V5-GW/lacZ, pENTR/U6, pLenti6-GW/U6-laminshrna and pLenti6/BLOCK-iT-DEST (Invitrogen). These vectors and others are available from commercial suppliers.

It is preferred that the antisense oligonucleotide or siRNA molecule be constructed and arranged so as to bind selectively with the target under physiological conditions, i.e., to hybridize substantially more to the target sequence than to any other sequence in the target cell under physiological conditions. One of skill in the art can easily choose and synthesize any of a number of appropriate antisense or siRNA molecules for use in accordance with the present invention. In order to be sufficiently selective and potent for inhibition, such antisense oligonucleotides should comprise at least 10 and, more preferably, at least 15 consecutive bases which are complementary to the target, although in certain cases modified oligonucleotides as short as 7 bases in length have been used successfully as antisense oligonucleotides (Wagner et al., Nature Biotechnol. 14:840-844, 1996). Most preferably, the antisense oligonucleotides comprise a complementary sequence of 20-30 bases. For siRNA molecules, it is preferred that the molecules be 21-23 nucleotides in length, with a 3′ 2 nucleotide overhang, although shorter and longer molecules and molecules without overhangs are also contemplated as useful in accordance with the invention.

The antisense is targeted, preferably, to sites in which mRNA secondary structure is not expected (see, e.g., Sainio et al., Cell Mol. Neurobiol. 14(5):439-457, 1994) and at which polypeptides are not expected to bind. Other methods for selecting preferred siRNA sequences are known to those of skill in the art (e.g., the “siRNA Selection Program” of the Whitehead Institute for Biomedical Research (2003)).

In one set of embodiments, the antisense oligonucleotides or siRNA molecules of the invention may be composed of “natural” deoxyribonucleotides, ribonucleotides, or any combination thereof. That is, the 5′ end of one native nucleotide and the 3′ end of another native nucleotide may be covalently linked, as in natural systems, via a phosphodiester internucleoside linkage. These oligonucleotides may be prepared by art recognized methods which may be carried out manually or by an automated synthesizer. They also may be produced recombinantly by vectors, including in situ.

In preferred embodiments, however, the antisense oligonucleotides or siRNA molecules of the invention also may include “modified” oligonucleotides. That is, the oligonucleotides may be modified in a number of ways which do not prevent them from hybridizing to their target but which enhance their stability or targeting or which otherwise enhance their therapeutic effectiveness.

The term “modified oligonucleotide” as used herein describes an oligonucleotide in which (1) at least two of its nucleotides are covalently linked via a synthetic internucleoside linkage (i.e., a linkage other than a phosphodiester linkage between the 5′ end of one nucleotide and the 3′ end of another nucleotide) and/or (2) a chemical group not normally associated with nucleic acids has been covalently attached to the oligonucleotide. Preferred synthetic internucleoside linkages are phosphorothioates, alkylphosphonates, phosphorodithioates, phosphate esters, alkylphosphonothioates, phosphoramidates, carbamates, carbonates, phosphate triesters, acetamidates, carboxymethyl esters and peptides.

The term “modified oligonucleotide” also encompasses oligonucleotides with a covalently modified base and/or sugar. For example, modified oligonucleotides include oligonucleotides having backbone sugars which are covalently attached to low molecular weight organic groups other than a hydroxyl group at the 3′ position and other than a phosphate group at the 5′ position. Thus modified oligonucleotides may include a 2′-O-alkylated ribose group. In addition, modified oligonucleotides may include sugars such as arabinose instead of ribose. The present invention, thus, contemplates pharmaceutical preparations containing modified antisense molecules that are complementary to and hybridizable with, under physiological conditions, the gene product of interest, together with pharmaceutically acceptable carriers.

The preparations of the invention, such as a population of DA neurons, precursors or progenitors, a composition that modulates expression of one or more dopamine neuron determinant gene products, or an activator of one or more dopamine neuron determinant gene products, are administered in effective amounts. An effective amount is that amount of a pharmaceutical preparation that alone, or together with further doses, produces the desired response. In the case of treating a condition characterized by neurodegeneration, the desired response is slowing neurodegeneration or increasing the presence of neurons, preferably to a level which is within a normal range. The responses can be monitored by routine methods in the art, such as standard clinical assessments of neurological function and diagnostic methods provided by the invention.

Such amounts will depend, of course, on the particular condition being treated, the severity of the condition, the individual patient parameters including age, physical condition, size and weight, the duration of the treatment, the nature of concurrent therapy (if any), the specific route of administration and like factors within the knowledge and expertise of the health practitioner. It is preferred generally that a maximum dose be used, that is, the highest safe dose according to sound medical judgment. It will be understood by those of ordinary skill in the art, however, that a patient may insist upon a lower dose or tolerable dose for medical reasons, psychological reasons or for virtually any other reasons.

For cell transplantation therapies, it is expected that a single dose of cells will be effective to provide sufficient DA neurons to ameliorate the condition being treated. However, administration of multiple doses of cells also is contemplated. The dose of cells administered will be established by the practitioner conducting the treatment. Doses of 10⁴-10⁸ cells are contemplated as preferred, although greater or lesser doses can be administered depending on the conditions for determining effective amounts as described above, as well as the replicative potential of the cells.

Generally, doses of active compounds would be from about 0.01 ng/kg per day to 1000 mg/kg per day. It is expected that doses ranging from 50 μg-500 mg/kg will be suitable and in one or several administrations per day. Lower doses can result from other forms of administration, such as intravenous administration. In the event that a response in a subject is insufficient at the initial doses applied, higher doses (or effectively higher doses by a different, more localized delivery route) may be employed to the extent that patient tolerance permits. Multiple doses per day are contemplated to achieve appropriate systemic levels of compound, although fewer doses typically will be given when compounds are prepared as slow release or sustained release medications.

When administered, the pharmaceutical preparations of the invention are applied in pharmaceutically-acceptable amounts and in pharmaceutically-acceptably compositions. Such preparations may routinely contain salts, buffering agents, preservatives, compatible carriers, and optionally other therapeutic agents. When used in medicine, the salts should be pharmaceutically acceptable, but non-pharmaceutically acceptable salts may conveniently be used to prepare pharmaceutically-acceptable salts thereof and are not excluded from the scope of the invention. Such pharmacologically and pharmaceutically-acceptable salts include, but are not limited to, those prepared from the following acids: hydrochloric, hydrobromic, sulfuric, nitric, phosphoric, maleic, acetic, salicylic, citric, formic, malonic, succinic, and the like. Also, pharmaceutically-acceptable salts can be prepared as alkaline metal or alkaline earth salts, such as sodium, potassium or calcium salts.

Compositions that modulate expression of one or more dopamine neuron determinant gene products or activators of one or more dopamine neuron determinant gene products useful according to the invention may be combined, optionally, with a pharmaceutically-acceptable carrier. The term “pharmaceutically-acceptable carrier” as used herein means one or more compatible solid or liquid fillers, diluents or encapsulating substances which are suitable for administration into a human. The term “carrier” denotes an organic or inorganic ingredient, natural or synthetic, with which the active ingredient is combined to facilitate the application. The components of the pharmaceutical compositions also are capable of being co-mingled with the molecules of the present invention, and with each other, in a manner such that there is no interaction which would substantially impair the desired pharmaceutical efficacy.

The pharmaceutical compositions may contain suitable buffering agents, including: acetic acid in a salt; citric acid in a salt; and phosphoric acid in a salt.

The pharmaceutical compositions also may contain, optionally, suitable preservatives, such as: benzalkonium chloride; chlorobutanol; parabens and thimerosal.

A variety of administration routes are available. The particular mode selected will depend, of course, upon the particular cells or compound selected, the severity of the condition being treated and the dosage required for therapeutic efficacy. The methods of the invention, generally speaking, may be practiced using any mode of administration that is medically acceptable, meaning any mode that produces effective levels of the cells or active compounds without causing clinically unacceptable adverse effects. Such modes of administration include oral, rectal, topical, nasal, intradermal, or parenteral routes. The term “parenteral” includes subcutaneous, intravenous, intrathecal, intracranial, intramuscular, as a bolus or as an infusion.

The pharmaceutical compositions may conveniently be presented in unit dosage form and may be prepared by any of the methods well-known in the art of pharmacy. All methods include the step of bringing the active agent into association with a carrier which constitutes one or more accessory ingredients. In general, the compositions are prepared by uniformly and intimately bringing the active compound into association with a liquid carrier, a finely divided solid carrier, or both, and then, if necessary, shaping the product.

Compositions suitable for oral administration may be presented as discrete units, such as capsules, tablets, lozenges, each containing a predetermined amount of the active compound. Other compositions include suspensions in aqueous liquids or non-aqueous liquids such as a syrup, elixir or an emulsion.

Compositions suitable for parenteral administration conveniently comprise a sterile aqueous preparation of a composition that modulates expression of one or more dopamine neuron determinant gene products or an activator of one or more dopamine neuron determinant gene products, which is preferably isotonic with the blood of the recipient. This aqueous preparation may be formulated according to known methods using suitable dispersing or wetting agents and suspending agents. The sterile injectable preparation also may be a sterile injectable solution or suspension in a non-toxic parenterally-acceptable diluent or solvent, for example, as a solution in 1,3-butane diol. Among the acceptable vehicles and solvents that may be employed are water, Ringer's solution, and isotonic sodium chloride solution. In addition, sterile, fixed oils are conventionally employed as a solvent or suspending medium. For this purpose any bland fixed oil may be employed including synthetic mono- or di-glycerides. In addition, fatty acids such as oleic acid may be used in the preparation of injectables. Carrier formulation suitable for oral, subcutaneous, intravenous, intrathecal, intramuscular, etc. administrations can be found in Remington's Pharmaceutical Sciences, Mack Publishing Co., Easton, Pa.

Other delivery systems can include time-release, delayed release or sustained release delivery systems. Such systems can avoid repeated administrations of the active compound, increasing convenience to the subject and the physician. Many types of release delivery systems are available and known to those of ordinary skill in the art. Use of a long-term sustained release implant may be desirable. Long-term release, are used herein, means that the implant is constructed and arranged to delivery therapeutic levels of the active ingredient for at least 30 days, and preferably 60 days. Long-term sustained release implants are well-known to those of ordinary skill in the art and include some of the release systems described above.

Also provided are methods for identifying DA neuron progenitor cells, as well as the cells so identified. Such methods include determining the expression in a cell of one or more Lmx1a, Msx1 and/or Msx2 gene products. The expression of the one or more gene products indicates that the cell is a DA neuron progenitor cell. The DA neuron progenitor cells can be embryonic progenitor cells.

The expression of the gene products (e.g., RNAs, proteins) can be determined using any method of assaying nucleic acid or the polypeptide products thereof known to one of ordinary skill in the art. For example, expression of nucleic acid gene products can be assayed by reverse transcriptase PCR (RT-PCR) or nucleic acid hybridization, such as various nucleic acid blotting methods known in the art. For determining expression of protein gene products, methods such as those including binding of an antibody or antibody fragment to the protein are contemplated.

Other methods for isolating DA neuron progenitor cells include contacting a population of cells with a reagent that binds to of one or more Lmx1a, Msx1 and/or Msx2 gene products, and isolating cells that are bound by the reagent from the population. Cells isolated by such methods also are included in the invention. The DA neuron progenitor cells are, in some embodiments, embryonic progenitor cells.

According to such methods, the reagent that binds the one or more gene products is preferably labeled. In one set of embodiments, the one or more gene products is a protein, in which case the reagent preferably is an antibody or binding fragment thereof.

DA neurons also are provided, which are differentiated from the DA neuron progenitor cell described herein. Also provided are methods of treating a neurodegenerative disease or disorder, in which DA neurons or progenitor cells as described above are obtained and transplanted according to standard medical techniques into a subject having or suspected of having the neurodegenerative disease or disorder. The neurodegenerative disease or disorder preferably is Parkinson's disease, and preferably the DA neurons or progenitor cells are transplanted into the brain of the subject, more preferably into the striatum.

Still other methods for isolating DA neuron progenitor cells and/or DA neurons are contemplated. The DA neuron progenitor cells preferably are embryonic progenitor cells. In such methods, a population of cells is provided that includes a nucleic acid molecule that encodes a marker protein operatively linked to a Lmx1a, Msx1 and/or Msx2 promoter sequence. Cells that express the marker protein are isolated from the population.

The marker protein is detectable, such as a fluorescent protein (e.g., a green fluorescent protein) or a cell surface protein. The cells can be isolated by standard cell isolation methods, including fluorescence activated cell sorting and magnetic sorting, such as by using an antibody or binding fragment thereof to bind to the marker protein, wherein the antibody is linked to a magnetic particle or can be bound by a molecule linked to a magnetic particle. The population of cells is then subjected to a magnetic field to separate cells bound by the antibody or binding fragment thereof. Such methods are well known to those of skill in the art.

The cells isolated by these methods can be genetically engineered cells and preferably include, or are, stem cells, preferably embryonic stem cells, adult stem cell or genetically engineered stem cells.

DA neuron progenitor cells or DA neurons isolated by these method thus also are provided by the invention. The DA neuron progenitor cells can be differentiate to provide dopamine neurons. Further, these DA neuron progenitor cells and neurons can be used in methods of treating neurodegenerative diseases or disorders by transplanting the DA neurons or progenitor cells into a subject having or suspected of having the neurodegenerative disease or disorder. The neurodegenerative disease or disorder is preferably Parkinson's disease, although other uses for such methods in treating other neurodegenerative diseases will be apparent to one of ordinary skill in the art. Preferably the DA neurons or progenitor cells are transplanted into the brain of the subject, more preferably into the striatum.

The invention will be more fully understood by reference to the following examples. These examples, however, are merely intended to illustrate the embodiments of the invention and are not to be construed to limit the scope of the invention.

### EXAMPLES

### Materials and Methods

### Identification of Lmx1a and Msx1

Degenerated homeodomain primers (Dhawan et al., 1997) and RT-PCR were used to screen a cDNA library prepared from dissected mouse el 0.5 ventral midbrain tissue. Products were subcloned into pGemTA vector (Promega) and sequenced. For further studies, a full length cDNA of Msx1 was isolated from the mouse cDNA library using RT-PCR. Lmx1a was identified in a wide in situ hybridisaiton screen for transcription factors expressed in the developing CNS (J. Ericson).

DNA Constructs and siRNA

cDNAs encoding mouse homologues of Lmx1a (Riken clone AK044944), Lmx1b (Ma, Q), Msx1, ldbl (RXPD clone no. IRAKp96112317Q) and Grg4 (Muhr et al., 2001) were used. Constructs were inserted into pCAGGS, RCASBP (Briscoe et al., 2000), pECE (Ellis et al., 1986), CMXGAL4 or NesE vectors. NesE vector was generated by subcloning the nestin 1852 enhancer (Lothian and Lendahl, 1997) into a modified pBluescript vector carrying the PGK-neomycin gene. Full length Msx1 (amino acids 1-299) or Lmx1a (amino acids 1-382) mouse cDNAs were cloned into the NesE vector generating NesE-Msx1 and NesE-Lmx1a expression constructs. To generate ShhE-Msx1 transgenic mouse, Msx1 cDNA was inserted under a Shh promoter/enhancer region (Sexp5, D. Eppstein). Stealth siRNA for chick Lmx1a was designed using BLOCK-iT™ RNAi Designer provided by Invitrogen. The RNA duplex (5′ ACAGCGACGAAACCUCACUGAGCAA 3′) was used (Invitrogen). siRNA (5′ AACUGGACUUCCAGAAGAACA 3′) against human lamin was used as control (Dharmacon).

### In Ovo Electroporation

cDNA encoding Lmx1a and Msx1 in pCAGGS, RCASBP or pECE were microelectroporated at 1.5 μg/μl into the midbrain of HH (Hamburger and Hamilton, 1951) stage 10-12 chick embryos. siRNA was used at a final concentration of 2 μg/μl in H₂O with 1% Fast Green Dye.

### Neural Plate Explants

Neural plate explants (Yamada et al., 1993) corresponding to presumptive forebrain, midbrain and hindbrain regions (Muhr et al., 1997) were dissected from HH stage 6 chick embryos. The intermediate parts of each brain region not expressing Shh were further fine-dissected. Neural plate explants were cultured for 24-96 hr in the absence or presence of 15 nM Shh (R&D systems) (Ericson et al., 1996).

### COS-7 Cell Assays

COS-7 cells were transfected with 1.5 μg DNA. Plasmids: 100 ng MH100-tk-luciferase reporter plasmid (Perlmann and Jansson, 1995), 50 ng CMV-lacZ plasmid, 200 ng Gal4-expressing plasmid (Gal-only, Gal-Msx1, Gal-Msx1ΔEh1 or Gal-Lmx1a), 200-800 ng mGrg4, Ldb1 and/or pcDNA3 were cotransfected and harvested after 48 hr, and luciferase activity in individual transfections was compared with the value of the Gal-only control defined as 1 (˜50 000 luciferase units). Data points represent the average of at least three transfections +/−SD.

### Generation of NesE-Lmx1a and NesE-Msx1 Stable Mouse Embryonic Stem (ES) Cell Lines

E14.1 ES cells were routinely propagated on gelatinized culture dishes in DMEM culture medium (Invitrogen) supplemented with 2000 U/ml LIF (Chemicon), 10% Knockout serum replacement (KSR), 2% FCS, 0.1 mM non-essential amino acids, 1 mM pyruvate (all from Invitrogen) and 0.1 μM β2-mercaptoethanol (Sigma).

To generate stable ES cell lines, 2×10⁶ ES cells were nucleofected with 10 μg NesE-mMsx1 or NesE-mLmx1a linearized DNA according to the manufacturer's protocol (mouse ES nucleoporator kit, AMAXA, USA). Cells were re-plated on gelatinized 10 cm² dishes and the selection compound G418 was added 24 hours post-nucleofection. After eight days of selection, 15 NesE-Msx1 and 25 NesE-Lmx1a neomycin resistant colonies were isolated and tested for Lmx1a or Msx1 expression. ES cell lines stably expressing Msx1 and Lmx1a under control of the Nestin enhancer were identified based on activation of transgene expression during neural differentiation (NesE-Msx 1 #4, nesE-Lmx1a#19, and nesE-Lmx1a#17).

For in vitro differentiation, ES cells were washed once with PBS, plated on gelatinised dishes and grown in N2B27 differentiation medium (Ying et al., 2003) supplemented with 2 ng/ml bFGF (Invitrogen), 100 ng/ml FGF8 and various concentrations of SHH(R&D systems) for 0-28 days. Alternatively, after three days of differentiation cells were trypsinised and re-plated on poly-Ornithine and collagen (1 μg/ml) coated slides and grown for additional 5-20 days. Growth factors were removed after 3-4 days of differentiation. For immunohistochemistry, cells were fixed in 2% PFA in PBS for 15-40 minutes. Immunohistochemical localization of proteins was performed as described (see below; Briscoe et al, 2000). For statistical analysis either the number of positive colonies (where at least 40% of the cells expressed the relevant marker) or the number of double positive cells were counted. The data points represent the average of at least five independent experiments +/−SD.

### Immunohistochemistry and In Situ Hybridization

Immunohistochemical localization of proteins was performed essentially as described (Briscoe et al, 2000). Antibodies used: mouse Shh, Isl1, Otx1, Pax7, En1, LIM1/2, Msx1/2, Lmx1b, Ngn2, Nkx2.2 (Developmental Studies Hybridoma Bank), Tuj1 (Babco, USA), TH (MAB318, Chemicon), rabbit Lmx1a (German, M), Gbx2 (Edlund, T), Nkx2.2 (Jessell, T), Nurr1 (E-20, Santa Cruz Technology, Perlmann, T), GABA (Sigma), TH (PelFreeze), GFP (Molecular Probes), Pitx3 (Smidt et al., 2004) guinea pig Isl1/2, Nkx6.1 and Lmx1b (Jessell, T), rat DAT (Abcam). Whole mount in situ hybridization was performed essentially as described (Wilkinson, 1992). In situ hybridization on sections was performed as described in Briscoe et al., 2000 using chick TH (ChEST1010E8, MRC) or Nsg1 (Riken clone AK078054).

### Example 1

### Identification of DA Neuron Determinants

### Lmx1a and Msx1 are Expressed in Midbrain Dopamine Progenitor Cells

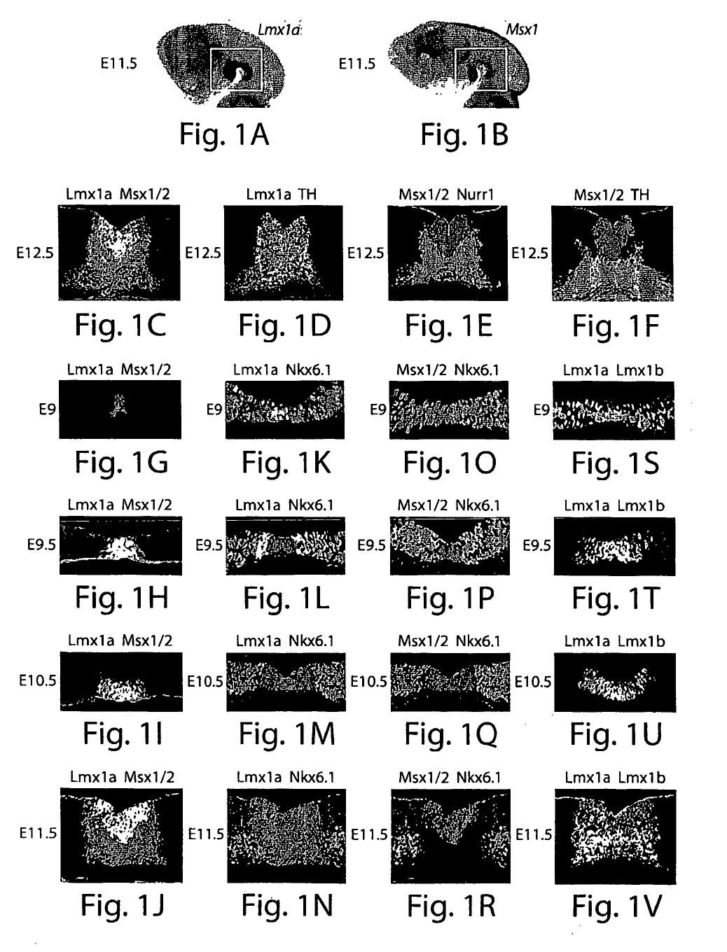

To identify homeodomain (HD) proteins expressed in DA progenitor cells in the ventral midbrain cDNA prepared from micro-dissected ventral midbrain tissue from embryonic day (E) 10.5 mouse embryos was used as a template to screen for HD-encoding transcripts by PCR. This approach, combined with a large scale in situ hybridization screen, identified Lmx1a and Msx1 as two transcription factors with relevant expression patterns. Whole-mount in situ hybridization at E11.5 showed that Lmx1a and Msx1 were both expressed in the ventral midbrain and at the caudal-most part of the diencephalon (FIG. 1A, B). We applied immunohistochemistry to further examine the expression of Lmx1a and Msx1 relative to defined markers of postmitotic dopaminergic (DA) neurons including Nurr1, Lmx1b and Pitx3 and tyrosine hydroxylase (TH), the rate limiting enzyme in DA synthesis (FIG. 1C-V; data not shown). At E12.5, a stage when DA neurons are actively generated, Lmx1a and Msx1 were co-expressed in DA progenitors located immediately above differentiating DA neurons (FIG. 1C-F). Expression of Lmx1a was maintained in postmitotic Nurr1₊/TH₊DA neurons (FIG. 1C, D) while Msx1 expression was exclusively confined to mitotic DA progenitors that had not yet initiated expression of Nurr1 and TH (FIG. 1C, E, F).

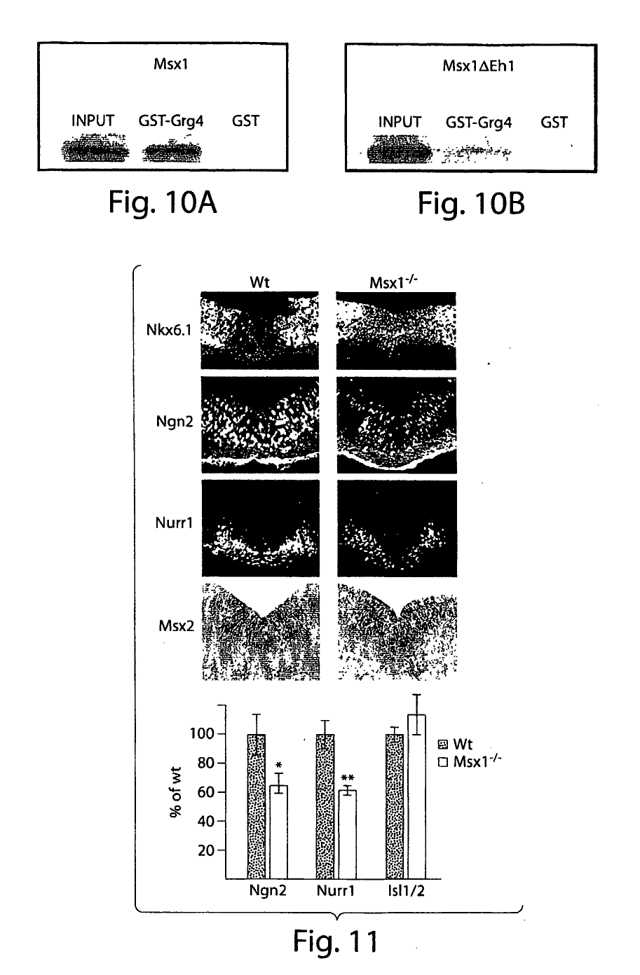

Lmx1a was expressed prior to Msx1 and was first detected in ventral midline cells at E9 (FIG. 1G). At this stage, Lmx1a was co-expressed with Nkx6.1, a HD protein that is broadly expressed in ventral progenitor cells (Vallstedt et al., 2001), including motor neuron progenitors located lateral to DA progenitors (FIG. 1K). From E9.5 and onwards, the expression of Nkx6.1 was progressively extinguished in presumptive DA progenitor cells (FIG. 1K-R). The downregulation of Nkx6.1 expression correlated precisely with the initiation of Msx1 expression at E9.5 (FIG. 10-IR). Msx2 was also expressed in a similar pattern as Msx1 but at much lower levels (see below).

Lmx1b is structurally related to Lmx1a and has previously been implicated in the maturation of DA neurons (Smidt et al., 2000). While expressed in ventral midbrain progenitors, Lmx1b was not restricted to DA progenitors at early developmental stages (FIG. 1S-1U). Also, expression of Lmx1b was down-regulated in DA progenitors at E11.5, approximately two days before the cessation of midbrain DA neuron generation while the expression of both Lmx1a and Msx1 were maintained throughout this period (FIG. 1V; data not shown). Thus, the expression patterns of Lmx1a and Msx1, but not that of Lmx1b, correlates with DA neuron specification. Taken together, an Lmx1a₊/Msx1₊/Nkx6.1-expression profile defines DA progenitor cells in the ventral midbrain.

### Sonic Hedgehog Induces Lmx1a and Msx1/2 Selectively in Midbrain Tissue In Vitro.

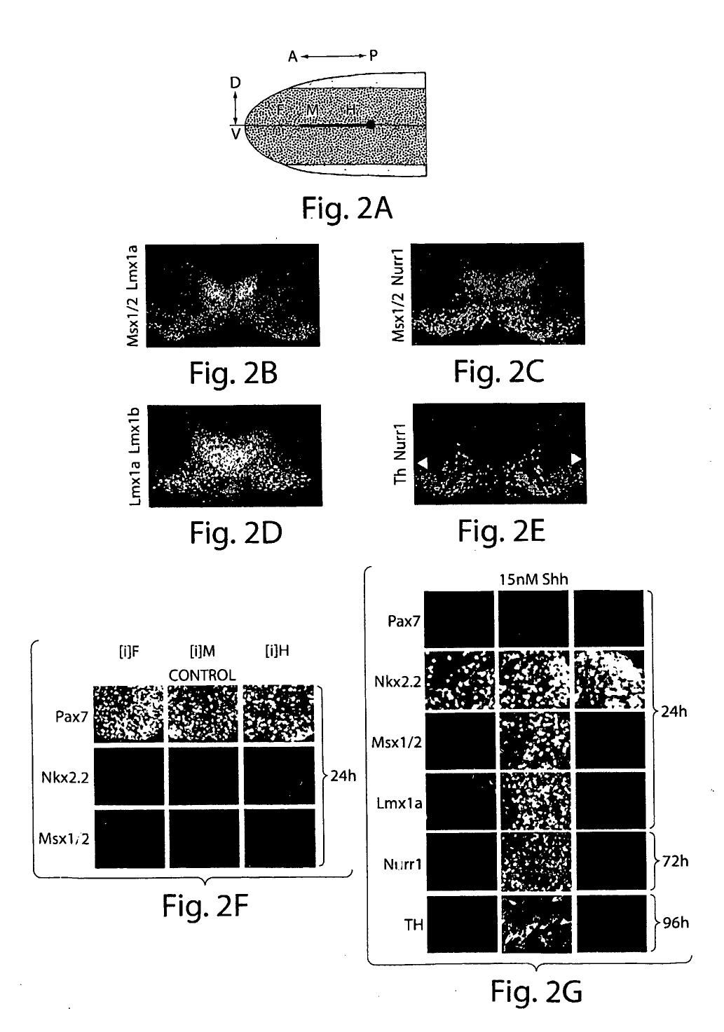

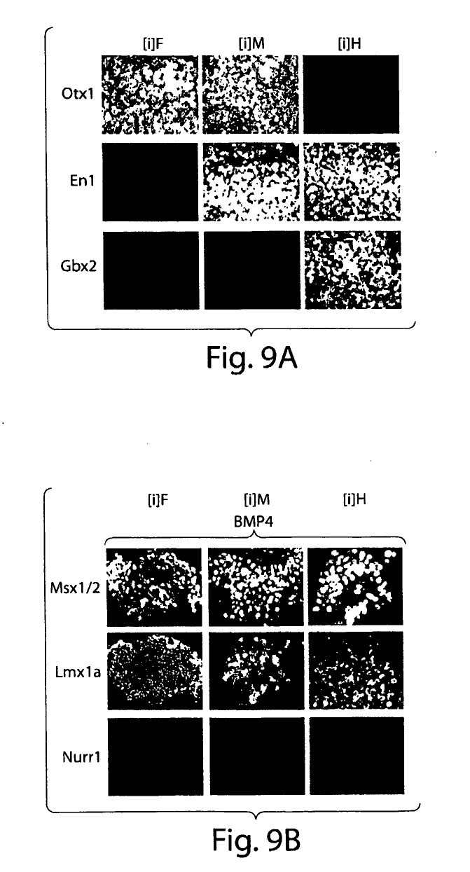

The vMB expression of Lmx1a and Msx1/2 implies that they are induced in response to ventrally derived Shh. To test this assumption, the ability of Shh to induce Lmx1a and Msx1/2 was examined in naïve intermediate neural plate explants isolated from the presumptive forebrain, midbrain and hindbrain levels of the chick neural plate ([i]F, [i]M, [i]H; FIG. 2A). Lmx1a and Msx1 were expressed in a similar fashion as in mouse embryos in the ventral midbrain of developing chick embryos (FIG. 2B-E). Expression of Otx1, En1 and Gbx2 was examined to monitor the anterior-posterior (AP) identities of explants in these experiments (FIG. 9A) (Martinez, 2001). In control [i]F, [i]M and [i]H explants grown for 24 hours in vitro, cells expressed the dorsal progenitor marker Pax7, but not Lmx1a, Msx1/2 or the ventral progenitor marker Nkx2.2 (FIG. 2F; data not shown) (Briscoe et al., 1999). Exposure of explants to 15 nM Shh resulted in ventralization of progenitor cell identity as determined by extinction of Pax7 expression and induction of Nkx2.2 (FIG. 2G). Notably, under this condition Lmx1a and Msx1/2 were induced only in midbrain explants and the induction of Lmx1a and Msx1/2 expression preceded the expression of the late DA cell markers Nurr1 and TH (FIG. 2G). Together, these data indicate that the ventral induction of Lmx1a and Msx1 depends on Shh signaling and that the ability of Shh to induce these factors is restricted along the AP axis.

### Lmx1a is Sufficient to Induce Dopamine Neurons In Vivo

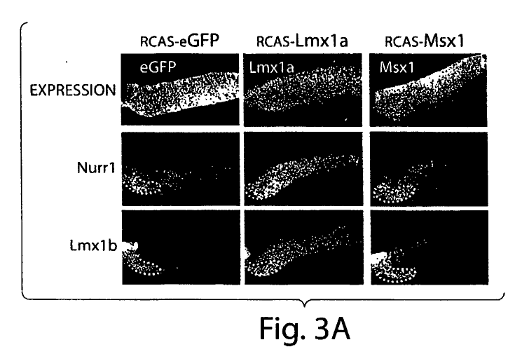

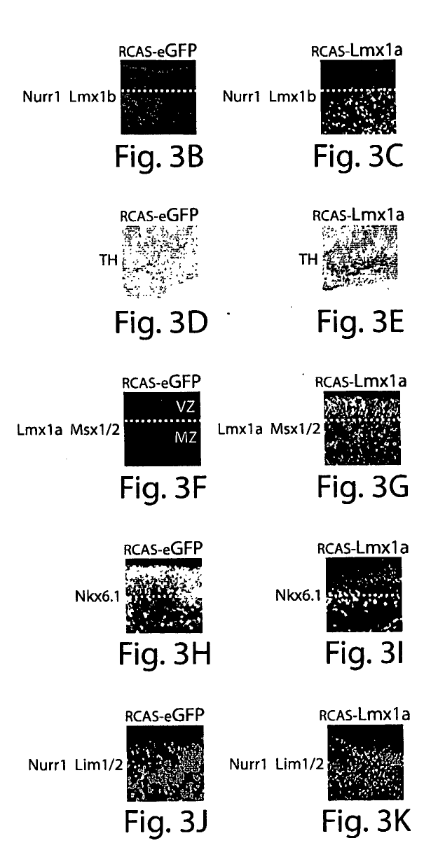

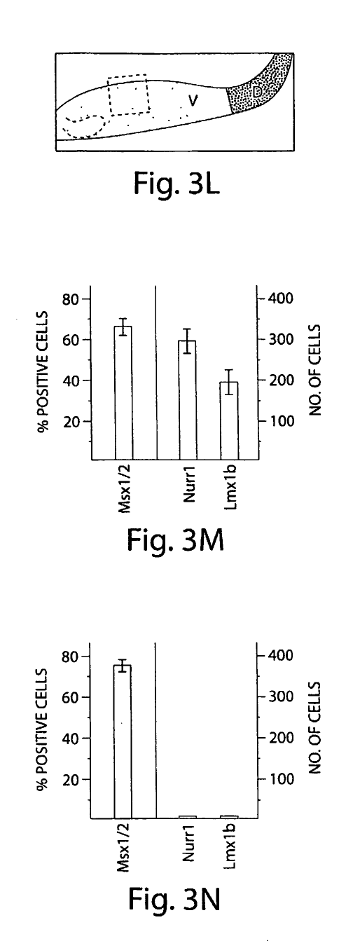

We next examined if Lmx1a and/or Msx1/2 can induce ectopic DA neurons in the chick midbrain. Lmx1a and Msx1 encoding cDNAs were inserted into retroviral RCAS vectors. Expression vectors were unilaterally transfected into the midbrain of HH stage 10 chick embryos by in ovo eletroporation and embryos were harvested and processed for analysis after 60-110 hours of incubation (Briscoe et al., 2000). Interestingly, forced expression of Lmx1a, but not Msx1, resulted in extensive induction of ectopic DA neurons in the ventral midbrain as indicated by the induction of Nurr1, Lmx1b and TH (FIG. 3A-E).

Induction of DA neurons by Lmx1a was preceded by a re-specification of progenitor cell identity as indicated by induction of Msx1 and suppression of Nkx6.1 expression in lateral progenitor cells (FIG. 3F-I). Also, the number of Lim1/2₊ neurons that are normally generated lateral to DA neurons were reduced, indicating that the induction of DA neurons by Lmx1a is accompanied by a concomitant loss of other ventral neuronal subtypes (FIG. 3J, K). Together, these data show that Lmx1a, but not Msx1, is sufficient to induce ectopic DA neurons in the midbrain. Moreover, in line with the sequential induction of Lmx1a and Msx1 observed in mice, these data provide evidence that Lmx1a activates the expression of Msx1 in DA progenitor cells.

Forced expression of Lmx1a in dorsal Pax7₊ progenitors did not result in the induction of Nurr1₊/Lmx1b₊ DA neurons despite its ability to induce Msx1 also in the dorsal midbrain (FIG. 3M, N) (Chizhikov and Millen, 2004b). Thus, since the competence of Lmx1a to induce DA neurons is limited to ventral progenitor cells, it is possible that ongoing Shh signaling or an as yet unidentified Shh-induced activity may act in parallel to Lmx1a in the induction of these cells. Alternatively, Shh signaling could repress a factor that suppresses the DA cell differentiation program in dorsal progenitor cells.

### Lmx1a is Required for the Generation of DA Neurons

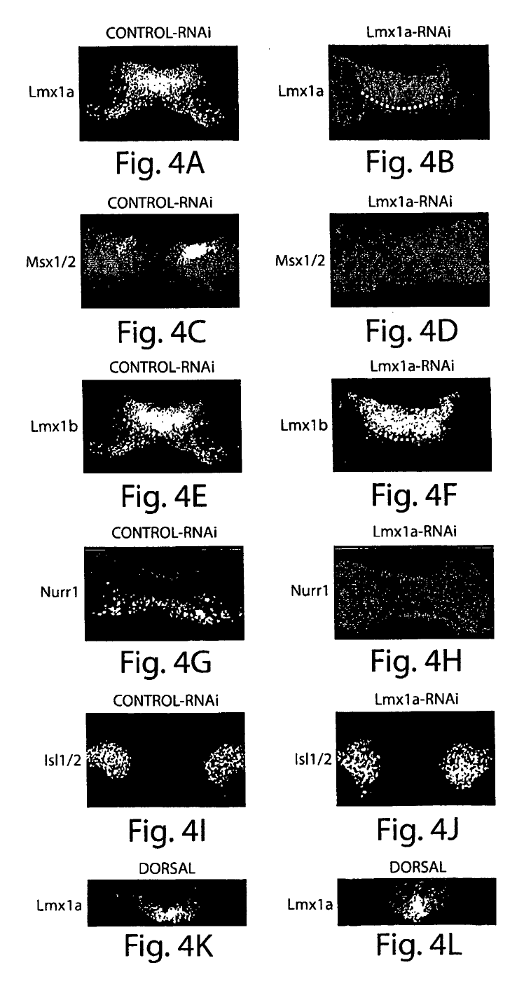

To test if Lmx1a is required for the generation of DA neurons we used RNAi to knock down Lmx1a in the chick ventral midbrain. While progenitor cell expression of Lmx1b was unaffected, essentially no expression of Lmx1a could be detected at 72 hours post-transfection (hpt) of an siRNA directed against the Lmx1a mRNA (FIG. 4B, F). In contrast, dorsal expression of Lmx1a was unaffected (FIG. 4L). Strikingly, the elimination of Lmx1a resulted in a drastic reduction of postmitotic Nurr1+/Lmx1b+DA neurons (FIG. 4D, F, H). In contrast, the generation of Isl1₊ motor neurons and Lim1/2₊ interneurons was unaffected (FIG. 4J; data not shown). Transfection of a control RNAi did not affect the expression of any analyzed marker (FIG. 4A, C, E, G, I, K). This loss-of-function experiment reveals a requirement for Lmx1a in the generation of DA neurons and also suggests that Lmx1b cannot compensate for the loss of Lmx1a.

Msx1 Represses Nkx6.1 and Acts Synergistically with Lnmx1a in the Induction DA Neurons.

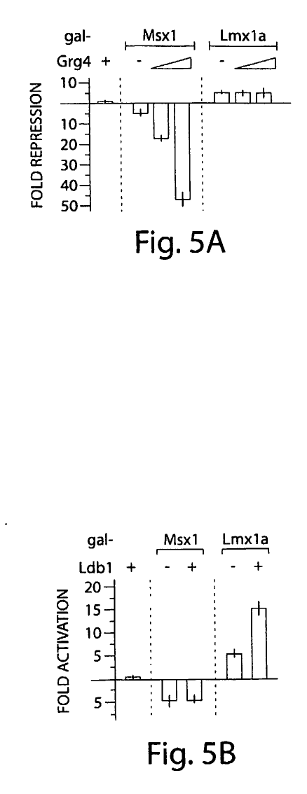

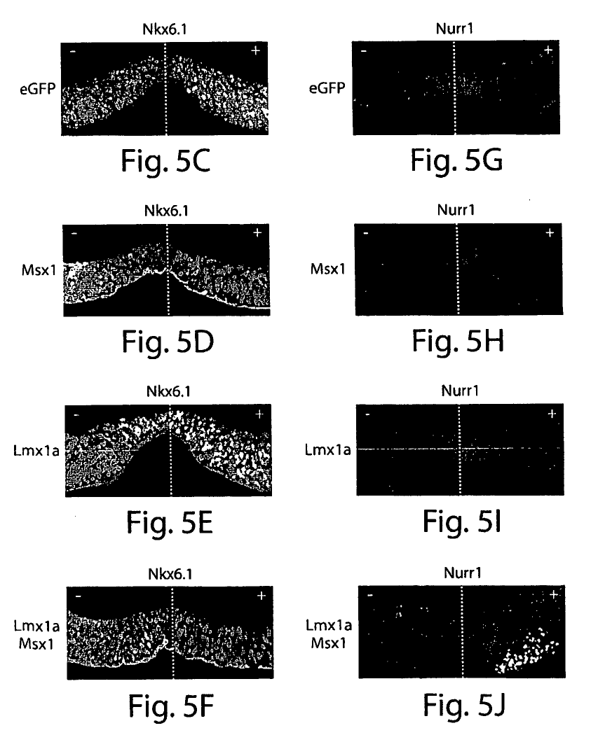

Our data show that Msx1 alone is not sufficient to induce DA neurons raising the issue of its function in DA progenitors (see FIG. 3A). Patterning HD proteins often function as Groucho/TLE-dependent transcriptional repressors (Muhr et al., 2001). Sequence analysis revealed that Msx1, but not Lmx1a, contained a putative Groucho/TLE-binding eh1-domain, supporting previous data showing that Msx1 can function as a transcriptional repressor (Catron et al., 1995; Muhr et al., 2001). Indeed, Msx1 functioned as a Groucho/TLE-dependent repressor in a reporter gene assay in transfected COS-7 cells (FIG. 5A). Msx1 could also interact with a bacterially produced GST-Groucho in vitro (FIG. 10). In contrast, in similar experiments Lmx1a did not repress transcription (FIG. 5A). Instead, Lmx1a functioned as a transcriptional activator whose activity was enhanced by co-transfection of the LIM-domain binding co-activator Ldb1 (FIG. 5B).

The gradual extinction of Nkx6.1 expression in DA progenitors correlates with the induction of Msx1 expression (see FIG. 1O-Q) and, therefore, the ability of Msx1 to repress expression of Nkx6.1 in vivo was examined. Transfected embryos were analyzed at 20 hpt, a time point when endogenous expression of Msx1 had not been initiated (FIG. 5C-J). To ensure rapid expression, we used an Msx1 expression vector driven by a hybrid cytomegalovirus enhancer/chicken beta-actin promoter (CAGG). Expression of Msx1 had essentially extinguished Nkx6.1 expression in ventral midbrain progenitors at 20 hpt (FIG. 5C-F). Nkx6.1 is required for the generation for motor neurons in the spinal cord (Sander et al., 2000; Vallstedt et al., 2001), and we noted that forced expression of Msx1 reduced the number of Is 1/2₊ motor neurons in the ventral midbrain (data not shown). Together these data show that Msx1 is a repressor of Nkx6.1 expression and suggest that Msx1 functions to suppress alternative ventral cell fates in the DA progenitor domain.

As shown above, Lmx1a can also repress Nkx6.1 expression (FIG. 3I). However, since Lmx1a induces Msx1 it appears likely that Lmx1a-mediated suppression of Nkx6.1 is indirect. Indeed, transfection CAGGS-Lmx1a did not result in the repression of Nkx6.1 at 20hpt, a timepoint when Lmx1a had not yet induced Msx1 (FIG. 5E; data not shown). These data indicate that Lmx1a indirectly suppresses the expression of Nkx6.1 via the induction of Msx1.

We next considered the consequences of combined Lmx1a and Msx1 expression in ventral midbrain progenitors. Remarkably, co-transfection of CAGGS-Msx1 and CAGGS-Lmx1a induced Nurr1₊ cells already at 60 hpt, a stage when Nurr1₊ cells had not yet been generated on the untransfected control side (FIG. 5J). Forced expression of Msx1 or Lmx1a alone had no apparent effect on the induction of Nurr1₊ cells at this stage (FIG. 5H, I). Thus, Msx1 appears to influence the timing of DA neuron induction when co-expressed with Lmx1a.

### Msx1 Suppresses Floor Plate Characteristics and Induce Ngn2 and Pan-Neuronal Differentiation in the Ventral Midbrain.

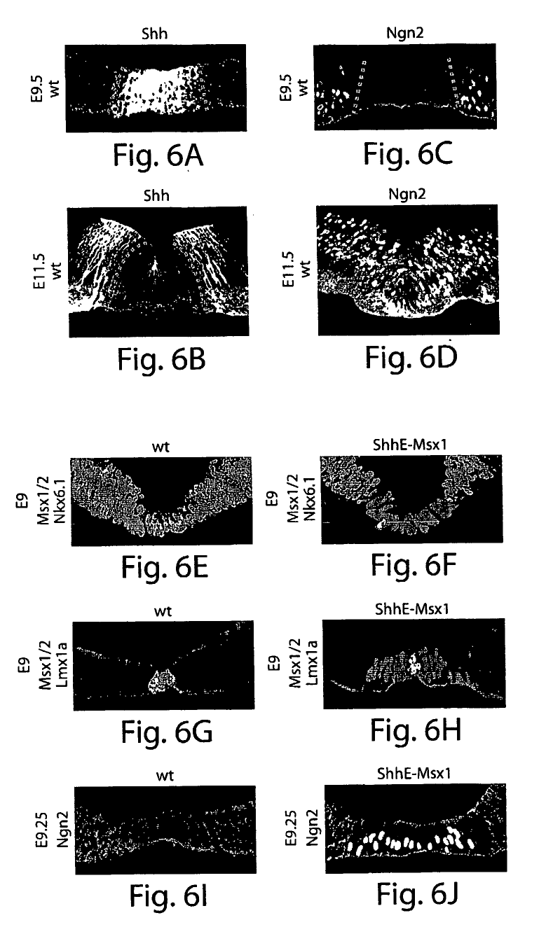

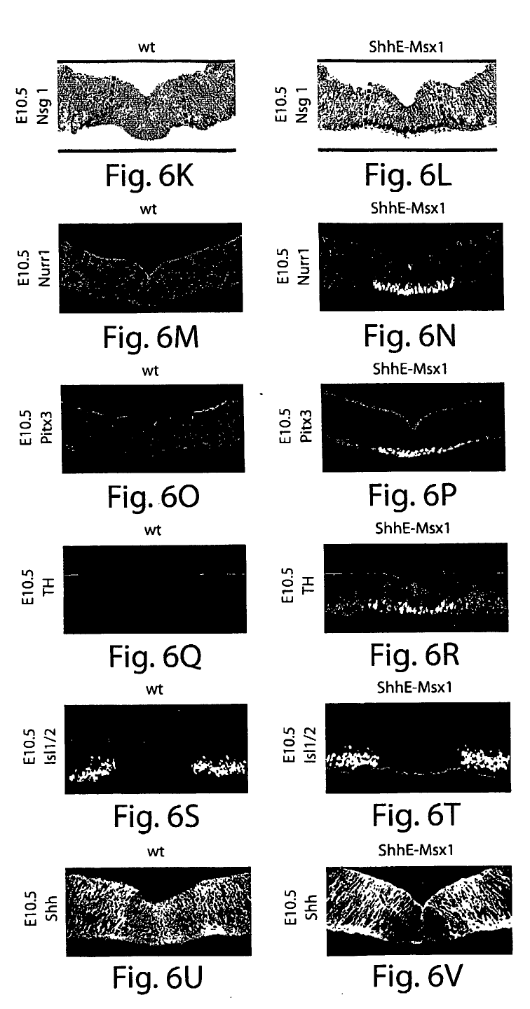

The ventral midline of the midbrain is occupied by glial-like Shh, floor plate cells prior to the generation of DA neurons (Placzek and Briscoe, 2005; Placzek et al., 1993). Thus, the generation of DA neurons must be preceded by a glial-to-neuronal conversion. Consistent with such a transition, the proneural basic helix-loop-helix protein Ngn2 (Bertrand et al., 2002) begins to be expressed at the ventral midline of the midbrain at around E10.75, and the expression of Shh becomes down-regulated in DA progenitors at E11.5 (FIG. 6A-D). Since our experiments indicate that Msx1 influences the timing of DA neuron production, it seemed possible that Msx1 mediates the suppression of floor plate characteristics and induces neuronal differentiation in ventral midline cells. To test this possibility, Msx1 was prematurely expressed in transgenic mice using a Shh enhancer (ShhE) (Epstein et al., 1999) which is active at least 24 hours before induction of endogenous Msx1 expression in the ventral midbrain. Strikingly, premature activation of Msx1 resulted in rapid extinction of Nkx6.1 expression and a marked induction of Ngn2 expression in ventral midline cells already at E9-9.25 (FIG. 6E-J). Notably, premature Msx1 expression did not affect the induction of Lmx1a (FIG. 6G, H). The early activation of Ngn2 was followed by premature induction of the pan-neuronal marker Nsg1 and DA specific markers Nurr1, Pitx3 and TH at E10.5 (FIG. 6K-R). Moreover, the premature induction of DA neurons in ShhE-Msx1 transgenic embryos was associated with a marked down-regulation of Shh expression in ventral midline cells already at E10.5 (FIG. 6U, V). In conclusion, premature induction of Msx1 results in upregulation of Ngn2 expression, a loss of floor-plate characteristic and premature induction of DA neurons suggesting that Msx1 controls the timing of DA cell neurogenesis.

To examine if Msx1 is also required for the proper generation of DA neurons we analyzed Msx1 knock-out mouse embryos (Houzelstein et al., 1997). At E11.5 Nkx6.1 expression was not extinguished in the ventral midline of Msx1 mutant embryos (FIG. 11). The progenitor cell expression of Msx2 and Lmx1a appeared unaffected by the loss of Msx1 function (FIG. 11; data not shown). Moreover, as compared to littermate controls, a 40% reduction in the number of Ngn2₊ progenitor cells and Nurr1₊ DA neurons was observed in mutants (FIG. 11). These data support our gain-of-function experiments and reveal a requirement of Msx1 for the proper production of DA neurons in the midbrain. Since DA neurons still can be generated in Msx1 mutants, however, it is feasible that Msx2 or another unidentified activity partly can compensate for the loss of Msx1 in this process.

### Example 2

### Engineering of DA Cells from Embryonic Stem Cells by Expression of Lmx1a and Msx1

We next examined the ability of Lmx1a, Msx1 and Lmx1b to induce DA cell differentiation in cultured mouse embryonic stem (ES) cells. In these experiments, cDNAs encoding Lmx1a, Msx 1, Lmx1b, and an eGFP control were inserted into expression vectors driven by a Nestin enhancer (NesE) (Lothian and Lendahl, 1997). The Nestin enhancer is active in neuronal progenitor cells, but not in undifferentiated ES cells or in postmitotic neurons (data not shown). Undifferentiated mouse ES cells were transfected and differentiated into Nestin+neuronal progenitors by plating on gelatin coated culture plates in medium containing FGF2, FGF8 and low concentration of Shh (1.7 nM) (Ying et al., 2003). Under these conditions, ES cells transfected with the NesE-eGFP generated neural progenitors that expressed En1/2 but not Pax7, Msx1 or Lmx1a (FIG. 7A, D). Thus, ES cells exposed to FGF2, FGF8 and low concentrations of Shh adopt an identity of ventral progenitors at the midbrain/rostral hindbrain level of the neuraxis. After 8 days of culture, most cells had differentiated into Tuj1₊ postmitotic neurons but none of these cells expressed TH (FIG. 7B).

Strikingly, transient transfection of a NesE-Lmx1a vector resulted in an extensive induction of Msx1/2₊ cells after 4 days and a robust generation of TuJ1+ neurons that co-expressed TH after 8 days of culture (FIG. 7A, B, D). Only rare TH₊ neurons could be detected after transfection with NesE-Lmx1b showing that Lmx1a is a much more potent inducer of TH₊ neurons (FIG. 7D). Importantly, essentially all Lmx1a-induced TH₊ neurons co-expressed Nurr1, Pitx3, En1/2, Lmx1a, Lmx1b and the dopamine transporter (DAT; FIG. 7C, E; data not shown), a profile of gene expression unique to midbrain dopamine neurons. Note that Lmx1a expression detected after 8 days corresponds to endogenous protein (FIG. 7C; data not shown). Also, essentially no Lmx1a-induced TH₊ cells were GABA₊, a neurotransmitter that is not expressed by midbrain DA neurons (FIG. 7C, E).

Consistent with our in vivo observations, Lmx1a was only competent to induce DA neurons in cells that had been ventralized by Shh. When ES cells were cultured in the absence of Shh, ES cell-derived neural progenitors expressed the dorsal progenitor cell marker Pax7 (FIG. 7A). Under these conditions NesE-Lmx1a induced Msx1/2 expression but no TH₊ neurons (FIG. 7A, B). Together, these data provide strong evidence that Lmx1a can induce the generation of bona fide midbrain DA neurons from ventralized ES-derived neural progenitor cells.

Although co-expression of TH, Nurr 1, Lmx1a, Lmx1b and En1/2 defines midbrain DA neurons, these factors are also individually expressed in other neuronal subtypes in the developing CNS (Asbreuk et al., 2002; Davis et al., 1991; Davis and Joyner, 1988; Failli et al., 2002; Zetterström et al., 1996a; Zetterström et al., 1996b). Previous studies have indicated that the exposure of ES cells to Shh and FGF8 can induce the generation of midbrain DA neurons (Barberi et al., 2003; Lee et al., 2000). We therefore examined the identity of neurons generated from ES cells exposed 15 nM Shh, a concentration sufficient to induce DA neurons in isolated primary neural plate tissue (FIG. 2). Importantly, in this condition Nurr1 and Lmx1b expression were induced but these transcription factors were not co-expressed in the same cells, and no ES cellderived TH₊ neurons were detected after 8 days of culture (FIG. 7E, F). After 12 days of culture, and consistent with previous studies, a subset of TuJ1₊ neurons had initiated TH expression (FIG. 7F) (Ying et al., 2003). However, the majority of these Shh-induced TH₊ cells co-expressed GABA and only rare TH₊ cells co-expressed Lmx1b or Pitx3 (FIG. 7E, F). Lmx1a transfected cells exposed to 15 nM Shh generated DA neurons with the same identity as cells exposed to 1.7 nM Shh (data not shown). Thus, while exposure of cells to high concentrations of Shh results in the generation of a few TH₊ cells after 12 days of culture, the vast majority of these cells do not coexpress other DA neuron markers indicating that Shh alone is insufficient to induce DA neurons with a “correct” midbrain identity. The reason why Shh alone is unable to induce DA neurons remains uncertain. However, it seems likely that ES cell-derived neural progenitors have a narrow “window of competence” for DA neuron generation, and that it is the rapid expression of Lmx1a after NesE-Lmx1a transfection that ensures robust induction of authentic DA neurons.

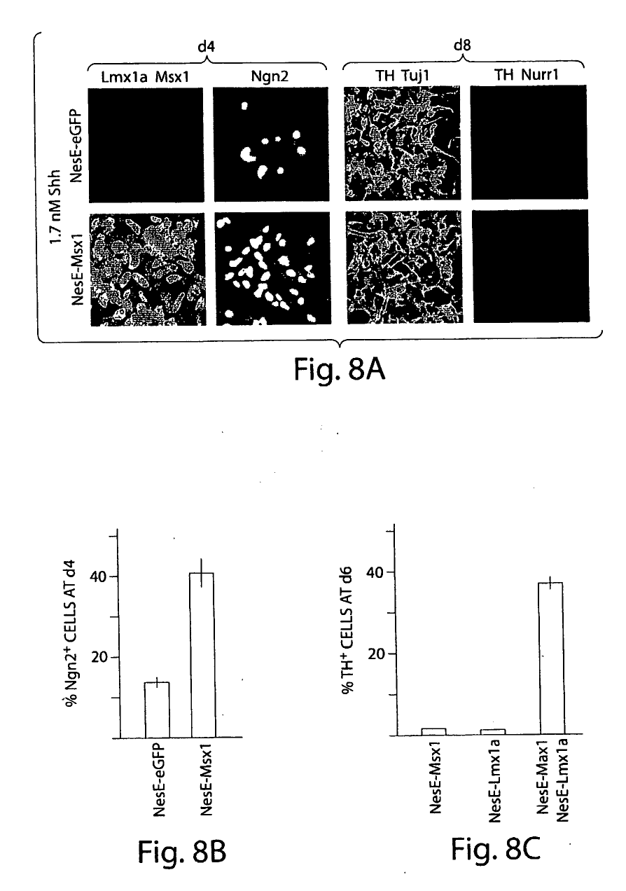

We also analyzed how transfection of NesE-Msx1 affected the process of ES cell differentiation. In vivo, Msx1 alone is unable to induce DA neurons, but is sufficient to induce Ngn2 expression. Similarly, Msx1 induced Ngn2, but not DA neurons, in ES cell-derived neural progenitors (FIG. 8A, B). Also consistent with in vivo data, Msx1 could influence the timing of DA neuron generation as indicated by a marked induction of TH₊ neurons already after 6 days of culture in NesE-Msx1 and NesE-Lmx1a co-transfected mES cells (FIG. 8C).

The prospect of using stem cell-derived DA neurons for transplantation in Parkinson's patients has emphasized the requirement to understand the normal pathway of DA neuron development. Here we show that Lmx1a is a transcriptional determinant of midbrain DA neurons. Msx1, which is rapidly induced by Lmx1a, suppresses floor-plate characteristics and induces pan-neuronal differentiation. Below we address the specific roles of Lmx1a and Msx1 in relation to DA neuron generation and discuss the potential of using these factors to control the generation of DA neurons from stem cells.

### Lmx1a is an Intrinsic Determinant of Midbrain DA Neurons.

Neuronal subtype specification has been extensively studied in the ventral spinal cord (Jessell, 2000; Lee and Pfaff, 2001). At this level, intrinsic cell fate determinants are typically HD proteins that are specifically expressed and function as Groucho/TLE-dependent transcriptional repressors (Muhr et al., 2001). A primary role of these transcription factors is to suppress the expression of other HD repressor proteins that are normally expressed in adjacent progenitor domains of the neural tube (Briscoe et al., 2000; Muhr et al., 2001). Accordingly, at a given position, one specific fate of differentiation is permitted through derepression, while alternative cell fates are actively suppressed (Muhr et al., 2001). The activation of specific downstream genes, in turn, appears to be mediated by broadly expressed transcriptional activators such as retinoic acid receptors, SP—1 and E2F (Lee et al., 2004; Muhr et al., 2001; Novitch et al., 2003). Transcriptional activators and repressors seem to be utilized in a somewhat different way in subtype specification of DA neurons. A marked difference between Lmx1a in midbrain DA cell progenitors and many previously defined HD determinants is that Lmx1a seems to function as a transcriptional activator. Suppression of alternative fates by Lmx1a appears to be mediated indirectly through the rapid induction of to Msx1 which, in turn, is a Groucho/TLE dependent HD repressor. Also, in contrast to the general transcriptional activators that seem to operate in the spinal cord it is feasible that Lmx1a, whose expression is maintained in differentiating postmitotic DA neurons, is itself a specific activator of downstream genes, including Msx1. Indeed, the inability of Msx1 to induce DA neuron differentiation outside of the endogenous DA progenitor domain directly supports this idea, and indicates that general activators alone are not sufficient to trigger DA cell differentiation.

In addition to Lmx1a, the structurally related protein Lmx1b is also expressed in DA progenitors (FIG. 1) (Asbreuk et al., 2002). However, the following observations suggest that Lmx1a and Lmx1b have distinct roles in the development of these cells: First, in contrast to Lmx1a, Lmx1b is not specifically expressed in DA progenitor cells and its expression is not maintained over the period of DA neuron generation (FIG. 1). Second, our Lmx1a-RNAi experiments provide evidence that Lmx1b cannot compensate for the loss of Lmx1a in the specification of DA neurons. Third, Lmx1a is a substantially more efficient inducer of DA neurons in ES cells as compared to Lmx1b. It is also notable that postmitotic DA markers are initially detected in Lmx1b null embryos, but are lost as development proceeds (Asbreuk et al., 2002). Thus, while we cannot exclude partial redundancy between these proteins, it seems likely that Lmx1b has a more profound role in differentiating postmitotic DA neurons. Msx1 coordinates patterning and pan-neuronal differentiation downstream of Lmx1a Our data indicate that Msx1 also induces cell-cycle exit and pan-neuronal differentiation through its ability to induce Ngn2. The activities of Msx1 in DA progenitors show analogy to the bHLH transcription factor Olig2, a repressor protein that induces Ngn2 expression and suppresses alternative cell fates in motor neuron progenitors in the spinal cord (Mizuguchi et al., 2001; Novitch et al., 2001; Zhou and Anderson, 2002). It is unclear how Msx1 and Olig2 can activate Ngn2 expression, but it seems plausible that Msx1 and Olig2 suppress the expression of a Ngn2 repressor, thus permitting activators to induce Ngn2 expression. Lmx1a would formally qualify as such an activator in the midbrain, but since Msx1 can induce Ngn2 expression in ES cell-derived Lmx1a neural progenitors (FIG. 7 and FIG. 8), more broadly expressed activators are likely to mediate this activity.

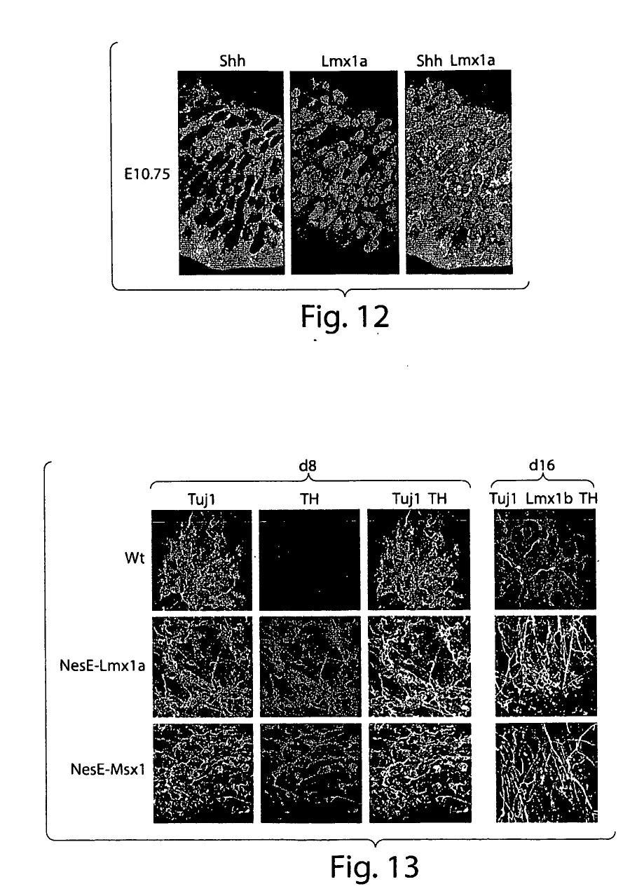

A unique aspect of DA neuron development is their generation at the ventral midline, which initially is occupied by glial-like floor plate cells (Placzek and Briscoe, 2005). Thus, in contrast to most other neurons, the birth of DA neurons must be preceded by a conversion of floor plate cells into neuronal progenitors. The early birth of DA neurons and the premature down-regulation of Shh expression at the ventral midline of ShhE-Msx1 transgenic mice indicate that Msx1 is intimately involved in this process. These data also favor a direct lineage relationship between floor plate cells and DA neurons, a conclusion that is directly supported by supported by the finding that Shh and Lmx1a are coexpressed in the ventral midline at early stages (FIG. 12). Thus, Msx1 does not simply induce pan-neuronal differentiation, but also seems to trigger a glial-to-neuronal switch in progenitor cell potential. Msx1 thereby sets the timing of DA neuron generation at the ventral midline, presumably through its ability to induce the expression of Ngn2.

### A Similar Pathway Controls the Generation of DA Neurons and Dorsal Roof Plate Cells

Considering the well established roles for Lmx1a, Lmx1b, Msx1 and Msx2 in dorsal cell fate specification and in particular roof plate cells (Bach et al., 2003; Chizhikov and Millen, 2004a; Chizhikov and Millen, 2004b; Millonig et al., 2000; Wang et al., 1996), it is striking that these four proteins also can be linked to the development of DA neurons at the ventral extreme of the midbrain. Since midbrain DA neurons have appeared relatively late in evolution as indicated by their absence in fish and certain amphibians, but presence in higher vertebrates (Gonzalez and Smeets, 1991), it is tempting to speculate that the emergence of this cell population involved a recruitment of an evolutionary ancient dorsal differentiation program (Arendt and Nubler-Jung, 1999). The ability of Lmx1a to induce roof plate cells or DA neurons would be determined by the dorsal-ventral identity of progenitor cells. Since DA neurons innervating the striatum are present in ventral diencephalon in fish, Lmx1a/b expressing DA neurons may also have been relocated from the diencephalon into the midbrain over evolutionary time. It is therefore noteworthy that ventral expression of Lmx1b.2 in Zebrafish is restricted to the diencephalon, while the closely related gene Lmx1b.1 also extends caudally into the midbrain (O'hara et al., 2005).

Lmx1a Directs Midbrain DA Neuron Differentiation from Mouse ES Cells

Ethical and practical issues associated with transplantation of fetal DA neurons to patients with Parkinson's disease has triggered intense interest in the possibility to use in vitro engineered stem cells as an unlimited cellular source for transplantation. A main objective of this research has been to investigate whether the identification of determinants that underly the specification of DA neurons during development can be exploited in a rational strategy to generate DA neurons from stem cells in vitro. In line with our in vivo analysis, Lmx1a functions as a DA neuron determinant in ES cells while Msx1 controls Ngn2 expression and pan-neuronal differentiation. Also, the DA neuron-inducing activity of Lmx1a is limited to ES derived cells that have been ventralized by Shh. These observations strongly suggest that the developmental pathway of DA neuron generation can be effectively recapitulated in differentiating ES cells in culture.

It is apparent that the correct identity of transplanted in vitro engineered DA neurons is a parameter that will critically influence the therapeutic potential of stem cell-derived DA neurons. Analyses of an array of midbrain DA markers at the single cell level unambiguously demonstrated that Lmx1a-expressing ES cells produced TH₊ neurons expressing an authentic midbrain identity (FIG. 7). In long-term cultures late markers of mature DA neurons were expressed, including the dopamine transporter and the vesicular monoamine transporter, providing additional evidence that these cells differentiate into bona fide midbrain DA neurons (FIG. 7; data not shown). In contrast, very few, if any, TH₊ neurons generated from mock transfected ES cells exposed to FGF8 and Shh expressed a correct midbrain DA neuron identity. A recent study using another established protocol for the generation of mES cell-derived TH₊ neurons is in line with our observations and showed that only a minority of TH₊ cells co-expressed the midbrain DA neuron marker Pitx3 (Zhao et al., 2004). These observations underscore the importance of validating the correct identity of stem cell-derived TH₊ cells by extensive marker analysis at the single cell level, and may provide an explanation why several transplantation studies using stem cell-derived neurons have met with limited success (Lindvall et al., 2004).

The identification of Lmx1a and Msx1 has shown that an increased understanding of the normal generation of DA neurons during development can enable the generation of bona fide DA neurons from stem cells. In previous studies, the developmental pathway of motor neuron generation has been recapitulated in a strategies to generate motor neurons from mES cells (Li et al., 2005; Wichterle et al., 2002). Up to 30% of cells differentiated into motor neurons when mES cells were exposed to Shh and retinoic acid showing that extrinsic signals can be sufficient to promote the generation of a clinically relevant cell type (Wichterle et al., 2002). While we cannot exclude that protocols using only extrinsic factors can be used for the efficient generation of midbrain DA neurons, our data imply that the combination of extrinsic signals and intrinsic determinants may be required as tools for the production of authentic DA neurons that ultimately can be used in the treatment of Parkinson's disease.

### Example 3

### Generation of NesE-Lmx1a and NesE-Msx1 Stable Mouse Embryonic Stem (ES) Cell Lines

ES cells were stably transformed with nestin promoter driven Lmx1a and Msx1, then differentiated, as described above.

The process of DA neuron differentiation from the stably transformed ES cells was extremely robust. Almost all (80-100%) Lmx1a-ES and approximately 50% of Msx1-ES cells differentiate into DA neurons.

The differentiated DA neurons are authentic as they expressed all analyzed markers that are expected to be expressed in bona fide DA neurons. The DA neurons were analyzed as described above, with results shown in FIG. 13. This confirmed the data from the transient experiments.

The DA neurons obtained from the stably transformed ES cells were transplanted into rat brains. These cells were integrated in a way that mimicked primary DA neurons.

### REFERENCES

Arendt, D., and Nubler-Jung, K. (1999). Comparison of early nerve cord development in insects and vertebrates. Development 126, 2309-2325.
Asbreuk, C. H., Vogelaar, C. F., Hellemons, A., Smidt, M. P., and Burbach, J. P. (2002). CNS expression pattern of Lmx1b and coexpression with ptx genes suggest functional cooperativity in the development of forebrain motor control systems. Mol Cell Neurosci 21, 410-420.
Bach, A., Lallemand, Y., Nicola, M. A., Ramos, C., Mathis, L., Maufras, M., and Robert, B. (2003). Msx1 is required for dorsal diencephalon patterning. Development 130, 4025-4036.
Barberi, T., Klivenyi, P., Calingasan, N.Y., Lee, H., Kawamata, H., Loonam, K., Perrier, A. L., Bruses, J., Rubio, M. E., Topf, N., et al. (2003). Neural subtype specification of fertilization and nuclear transfer embryonic stem cells and application in parkinsonian mice. Nat Biotechnol 21, 1200-1207.
Bertrand, N., Castro, D. S., and Guillemot, F. (2002). Proneural genes and the specification of neural cell types. Nat Rev Neurosci 3, 517-530.
Briscoe, J., Pierani, A., Jessell, T. M., and Ericson, J. (2000). A homeodomain protein code specifies progenitor cell identity and neuronal fate in the ventral neural tube. Cell 101, 435-445.
Briscoe, J., Sussel, L., Serup, P., Hartigan-O'Connor, D., Jessell, T. M., Rubenstein, J. L., and Ericson, J. (1999). Homeobox gene Nkx2.2 and specification of neuronal identity by graded Sonic hedgehog signalling. Nature 398, 622-627.
Catron, K. M., Zhang, H., Marshall, S. C., Inostroza, J. A., Wilson, J. M., and Abate, C. (1995). Transcriptional repression by Msx-1 does not require homeodomain DNA-binding sites. Mol Cell Biol 15, 861-871.
Chizhikov, V. V., and Millen, K. J. (2004a). Control of roof plate development and signaling by Lmx1b in the caudal vertebrate CNS. J Neurosci 24, 5694-5703. Chizhikov, V. V., and Millen, K. J. (2004b). Control of roof plate formation by Lmx1a in the developing spinal cord. Development 131, 2693-2705.
Davis, C. A., Holmyard, D. P., Millen, K. J., and Joyner, A. L. (1991). Examining pattern formation in mouse, chicken and frog embryos with an En-specific antiserum. Development 111, 287-298.
Davis, C. A., and Joyner, A. L. (1988). Expression patterns of the homeo box-containing genes En-1 and En-2 and the proto-oncogene int-1 diverge during mouse development. Genes Dev 2, 1736-1744.
Dhawan, R. R., Schoen, T. J., and Beebe, D. C. (1997). Isolation and expression of homeobox genes from the embryonic chicken eye. Mol V is 3, 7
Ellis, L., Clauser, E., Morgan, D. O., Edery, M., Roth, R. A., and Rutter, W. J. (1986). Replacement of insulin receptor tyrosine residues 1162 and 1163 compromises insulinstimulated kinase activity and uptake of 2-deoxyglucose. Cell 45, 721-732.
Epstein, D. J., McMahon, A. P., and Joyner, A. L. (1999). Regionalization of Sonic hedgehog transcription along the anteroposterior axis of the mouse central nervous system is regulated by Hnf3-dependent and -independent mechanisms. Development 126, 281-292.
Ericson, J., Morton, S., Kawakami, A., Roelink, H., and Jessell, T. M. (1996). Two critical periods of Sonic Hedgehog signaling required for the specification of motor neuron identity. Cell 87, 661-673.
Fulli, V., Bachy, I., and Retaux, S. (2002). Expression of the LIM-homeodomain gene Lmx1a (dreher) during development of the mouse nervous system. Mech Dev 118, 225-228.
Hamburger, H., and Hamilton, A. (1951). A series of normal stages in the development of the chick embryo. J Morphol 88, 49-92.
Haramis, A. G., Brown, J. M., and Zeller, R. (1995). The limb deformity mutation disrupts the SHH/FGF-4 feedback loop and regulation of 5′ HoxD genes during limb pattern formation. Development 121, 4237-4245.
Hynes, M., and Rosenthal, A. (1999). Specification of dopaminergic and serotonergic neurons in the vertebrate CNS. Curr Op Neurobiol 9, 26-36.
Jessell, T. M. (2000). Neuronal specification in the spinal cord: inductive signals and transcriptional codes. Nat Rev Genet 1, 20-29.
Lee, S. H., Lumelsky, N., Studer, L., Auerbach, J. M., and McKay, R. D. (2000). Efficient generation of midbrain and hindbrain neurons from mouse embryonic stem cells. Nat Biotechnol 18, 675-679.
Lee, S. K., Jurata, L. W., Funahashi, J., Ruiz, E. C., and Pfaff, S. L. (2004). Analysis of embryonic motoneuron gene regulation: derepression of general activators function in concert with enhancer factors. Development 131, 3295-3306.
Lee, S. K., and Pfaff, S. L. (2001). Transcriptional networks regulating neuronal identity in the developing spinal cord. Nat Neurosci 4 Suppl, 1183-1191.
Li, X. J., Du, Z. W., Zarnowska, E. D., Pankratz, M., Hansen, L. O., Pearce, R. A., and Zhang, S. C. (2005). Specification of motoneurons from human embryonic stem cells. Nat Biotechnol 23, 215-221.
Liem, K. F., Jr., Tremml, G., Roelink, H., and Jessell, T. M. (1995). Dorsal differentiation of neural plate cells induced by BMP-mediated signals from epidermal ectoderm. Cell 82, 969-979.
Lindvall, O., Kokaia, Z., and Martinez-Serrano, A. (2004). Stem cell therapy for human neurodegenerative disorders-how to make it work. Nat Med 10 Suppl, S42-50.
Lothian, C., and Lendahl, U. (1997). An evolutionarily conserved region in the second intron of the human nestin gene directs gene expression to CNS progenitor cells and to early neural crest cells. Eur J Neurosci 9, 452-462.
Martinez, S. (2001). The isthmic organizer and brain regionalization. Int J Dev Biol 45, 367-371.
Millonig, J. H., Millen, K. J., and Hatten, M. E. (2000). The mouse Dreher gene Lmx1a controls formation of the roof plate in the vertebrate CNS. Nature 403, 764-769.
Mizuguchi, R., Sugimori, M., Takebayashi, H., Kosako, H., Nagao, M., Yoshida, S., Nabeshima, Y., Shimamura, K., and Nakafuku, M. (2001). Combinatorial roles of olig2 and neurogenin2 in the coordinated induction of pan-neuronal and subtype-specific properties of motoneurons. Neuron 31, 757-771.
Muhr, J., Andersson, E., Persson, M., Jessell, T. M., and Ericson, J. (2001). Groucho-mediated transcriptional repression establishes progenitor cell pattern and neuronal fate in the ventral neural tube. Cell 104, 861-873.
Muhr, J., Jessell, T. M., and Edlund, T. (1997). Assignment of early caudal identity to neural plate cells by a signal from caudal paraxial mesoderm. Neuron 19, 487-502.
Novitch, B. G., Chen, A. I., and Jessell, T. M. (2001). Coordinate regulation of motor neuron subtype identity and pan-neuronal properties by the bHLH repressor Olig2. Neuron 31, 773-789.
Novitch, B. G., Wichterle, H., Jessell, T. M., and Sockanathan, S. (2003). A requirement for retinoic acid-mediated transcriptional activation in ventral neural patterning and motor neuron specification. Neuron 40, 81-95.
Nunes, I., Tovmasian, L. T., Silva, R. M., Burke, R. E., and Goff, S. P. (2003). Pitx3 is required for development of substantia nigra dopaminergic neurons. Proc Natl Acad Sci U S A 100, 4245-4250.
O'hara, F. P., Beck, E., Barr, L. K., Wong, L. L., Kessler, D. S., and Riddle, R. D. (2005). Zebrafish Lmx1b.1 and Lmx1b.2 are required for maintenance of the isthmic organizer. Development [Epub ahead of print].
Perlmann, T., and Jansson, L. (1995). A novel pathway for vitamin A signaling mediated by RXR heterodimerization with NGFI-B and NURR1. Genes Dev 9, 769-782.
Pierani, A., Moran-Rivard, L., Sunshine, M. J., Littman, D. R., Goulding, M., and Jessell, T. M. (2001). Control of interneuron fate in the developing spinal cord by the progenitor homeodomain protein Dbxl. Neuron 29, 367-384.
Placzek, M., and Briscoe, J. (2005). The floor plate: multiple cells, multiple signals. Nat Rev Neurosci 6, 230-240.
Placzek, M., Jessell, T. M., and Dodd, J. (1993). Induction of floor plate differentiation by contact-dependent, homeogenetic signals. Development 117, 205-218.
Placzek, M., Tessier-Lavigne, M., Yamada, T., Jessell, T., and Dodd, J. (1990). Mesodermal control of neural cell identity: floor plate induction by the notochord. Science 250, 985-988.
Ren, B., Chee, K. J., Kim, T. H., and Maniatis, T. (1999). PRD1-BF1/blimp1 repression is mediated by corepressors of the Groucho family of proteins. Genes Dev 13, 125-137.
Sander, M., Paydar, S., Ericson, J., Briscoe, J., Berber, E., German, M., Jessell, T. M., and Rubenstein, J. L. (2000). Ventral neural patterning by Nkx homeobox genes: Nkx6.1 controls somatic motor neuron and ventral interneuron fates. Genes Dev 14, 2134-2139.
Schaeren-Wiemers, N., and Gerfin-Moser, A. (1993). A single protocol to detect transcripts of various types and expression levels in neural tissue and cultured cells: in situ hybridization using digoxigenin-labelled cRNA probes. Histochemistry 100, 431-440.
Simon, H. H., Saueressig, H., Wurst, W., Goulding, M. G., and O'Leary, D. D. (1998). En-1 and En-2 control the fate of the dopaminergic neurons in the substantia nigra and ventral tegmentum. Eur J Neurosci 10, supplement 10, 389.
Smidt, M. P., Asbreuk, C. H., Cox, J. J., Chen, H., Johnson, R. L., and Burbach, J. P. (2000). A second independent pathway for development of mesencephalic dopaminergic neurons requires Lmx1b. Nat Neurosci 3, 337-341.
Smidt, M. P., Smits, S. M., and Burbach, J. P. (2004). Homeobox gene Pitx3 and its role in the development of dopamine neurons of the substantia nigra. Cell Tissue Res 318, 35-43.
Smidt, M. P., van Schaick, H. S., Lanctot, C., Tremblay, J. J., J., C. J., van der Kleij, A. A., Wolterink, G., Drouin, J., and Burbach, J. P. (1997). A homeodomain gene Ptx3 has highly restricted brain expression in mesencephalic dopaminergic neurons. Proc Natl Acad Sci USA 94, 13305-13310.
Vallstedt, A., Muhr, J., Pattyn, A., Pierani, A., Mendelsohn, M., Sander, M., Jessell, T. M., and Ericson, J. (2001). Different levels of repressor activity assign redundant and specific roles to Nkx6 genes in motor neuron and interneuron specification. Neuron 31, 743-755.
van den Munckhof, P., Luk, K. C., Step-Marie, L., Montgomery, J., Blanchet, P. J., Sadikot, A. F., and Drouin, J. (2003). Pitx3 is required for motor activity and for survival of a subset of midbrain dopaminergic neurons. Development 130, 2535-2542.
Wallen, A., and Perlmann, T. (2003). Transcriptional control of dopamine neuron development. Ann N Y Acad Sci 991, 48-60.
Wallen, A., Zetterstrom, R. H., Solomin, L., Arvidsson, M., Olson, L., and Perlmann, T. (1999). Fate of mesencephalic AHD2-expressing dopamine progenitor cells in NURR1 mutant mice. Exp Cell Res 253, 737-746.
Wang, W., Chen, X., Xu, H., and Lufkin, T. (1996). Msx3: a novel murine homologue of the Drosophila msh homeobox gene restricted to the dorsal embryonic central nervous system. Mech Dev 58, 203-215.
Wichterle, H., Lieberam, I., Porter, J. A., and Jessell, T. M. (2002). Directed differentiation of embryonic stem cells into motor neurons. Cell 110, 385-397.
Wilkinson, D. G. (1992). Whole-mount in situ hybridization of vertebrate embryos. In In situ hybridization. A practical approach., D. G.
Wilkinson, ed. (Oxford, IRL Press), pp. 75. Yamada, T., Pfaff, S. L., Edlund, T., and Jessell, T. M. (1993). Control of cell pattern in the neural tube: motor neuron induction by diffusible factors from notochord and floor plate. Cell 73, 673-686.
Ying, Q. L., Stpyridis, M., Griffiths, D., Li, M., and Smith, A. (2003). Conversion of embryonic stem cells into neuroectodermal precursors in adherent monoculture. Nat Biotechnol 21, 183-186.
Zetterström, R. H., Solomin, L., Jansson, L., Hoffer, B. J., Olson, L., and Perlmann, T. (1997). Dopamine neuron agenesis in Nurr1-deficient mice. Science 276, 248-250.
Zetterström, R. H., Solomin, L., Mitsiadis, T., Olson, L., and Perlmann, T. (1996a). Retinoid X receptor heterodimerization and developmental expression distinguish the orphan nuclear receptors NGFI-B, Nurr1, and Norl. Mol Endocrinol 10, 1656-1666.
Zetterström, R. H., Williams, R., Perlmann, T., and Olson, L. (1996b). Cellular expression of the immediate early transcription factors Nurr1 and NGFI-B suggests a gene regulatory role in several brain regions including the nigrostriatal dopamine system. Brain Res Mol Brain Res 41, 111-120.
Zhao, S., Maxwell, S., Jimenez-Beristain, A., Vives, J., Kuehner, E., Zhao, J., O'Brien, C., de Felipe, C., Semina, E., and Li, M. (2004). Generation of embryonic stem cells and transgenic mice expressing green fluorescence protein in midbrain dopaminergic neurons. Eur J Neurosci 19, 1133-1140.
Zhou, Q., and Anderson, D. J. (2002). The bHLH transcription factors OLIG2 and OLIG1 couple neuronal and glial subtype specification. Cell 109, 61-73.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

Furthermore, throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

Moreover, any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

Except where explicitly described otherwise, terms used in the singular also are meant to embrace the plural, and vice versa.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising”, “including”, “having”, “containing”, “composed of”, and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

Those skilled in the art will recognize, or be able to ascertain using no more than routine experimentation, many equivalents to the specific embodiments of the invention described herein. Such equivalents are intended to be encompassed by the following claims.

All references, including patent documents, disclosed herein are incorporated by reference in their entirety.

What is claimed is:

1. A cell that over-expresses Msx1, Msx2, Msx3, Lmx1a and/or Lmx1b,
optionally wherein the cell is isolated;
optionally wherein the cell is a stem cell or a non-neural, neural or neuronal progenitor or precursor, preferably wherein the stem cell is an embryonic stem cell;
optionally wherein the Msx1, Msx2, Msx3. Lmx1a and/or Lmx1b are expressed recombinantly, preferably wherein the recombinant expression is effectuated by operably linking the Msx1, Msx2, Msx3, Lmx1a and/or Lmx1b genes to an exogenous promoter and/or enhancer sequence, preferably wherein the exogenous promoter and/or enhancer sequence is contained in an expression vector that is introduced into the cell, preferably wherein the expression vector is introduction by transfection, infection, microinjection, electroporation or recombination, preferably wherein the promoter is from the herpes simplex virus thymidine kinase gene or the Rous sarcoma virus LTR, and wherein the enhancer is an enhancer from the nestin gene or a Sonic hedgehog (Shh) enhancer;
optionally wherein the cell is human;
optionally wherein the Msx1, Msx2, Msx3, Lmx1a and/or Lmx1b is encoded by a nucleic acid molecule comprising SEQ ID NO:1 encoding Msx1, SEQ ID NO:3 encoding Msx2, SEQ ID NO:5 encoding Msx3, SEQ ID NO:7 encoding Lmx1a, SEQ ID NO:9 encoding Lmx1b, a coding sequence thereof, or a fragment or derivative thereof that contributes to inducing differentiation of the cell into a DA neuron; optionally wherein the cell, when cultured, differentiates into DA neurons.

2-12. (canceled)

13. An cell line comprising the cells of claim 1.

14. An isolated population of cells or culture of cells as claimed in claim 1,
optionally further comprising a carrier, preferably wherein the carrier is a pharmaceutically acceptable carrier or a cell freezing medium or cell storage medium.

15.-18. (canceled)

19. A dopamine (DA) neuron differentiated from a cell by over-expression of Msx1, Msx2, Msx3, Lmrx1a and/or Lmx1b in the cell,
optionally wherein the DA neuron is isolated;
optionally wherein the cell is a stem cell, or a non-neural, neural and/or neuronal progenitor or precursor, preferably wherein the stem cell is an embryonic stem cell;
optionally wherein the Msx1, Msx2, Msx3, Lmx1a and/or Lmx1b are expressed recombinantly, preferably wherein the recombinant expression is effectuated by operably linking the Msx1, Msx2, Msx3, Lmx1a and/or Lmx1b genes to an exogenous promoter and/or enhancer sequence, preferably wherein the exogenous promoter and/or enhancer sequence is contained in an expression vector that is introduced into the DA neuron, preferably wherein the expression vector is introduction by transfection, infection, microinjection, electroporation or recombination, preferably wherein the promoter is from the herpes simplex virus thymidine kinase gene or the Rous sarcoma virus LTR, and wherein the enhancer is an enhancer from the nestin gene or a Sonic hedgehog (Shh) enhancer;
optionally wherein the DA neuron is human;
optionally wherein the Msx1, Msx2, Msx3, Lmx1a and/or Lmx1b is encoded by a nucleic acid molecule comprising SEQ ID NO: 1 encoding Msx1, SEQ ID NO:3 encoding Msx2, SEQ ID NO:5 encoding Msx3, SEQ ID NO:7 encoding Lmx1a, SEQ ID NO:9 encoding Lmx1b, a coding sequence thereof, or a fragment or derivative thereof that contributes to inducing differentiation of the cell into a DA neuron.

20.-29. (canceled)

30. A DA neuron cell line comprising the DA neurons of claim 19.

31. An isolated population or culture of DA neurons as claimed in claim 19,
optionally further comprising a carrier, preferably wherein the carrier is a pharmaceutically acceptable carrier, preferably wherein the carrier is a cell freezing medium or cell storage medium.

32.-35. (canceled)

36. A method for producing dopamine (DA) neurons, comprising
increasing the expression of Msx1, Msx2, Msx3, Lmx1a and/or Lmx1b in a cell, and
culturing the cell having increased expression of Msx1, Msx2, Msx3, Lmx1a and/or Lmx1b under conditions and for a time sufficient to permit differentiation of the cell to a DA neuron,
optionally wherein the cell is isolated;
optionally wherein the cell is a stem cell, or a non-neural, neural and/or neuronal progenitor or precursor, preferably wherein the stem cell is an embryonic stem cell;
optionally further comprising isolating the DA neuron;
optionally wherein the expression of Msx1, Msx2, Msx3, Lmx1a and/or Lmx1b is increased by over-expressing one or more nucleic acids that encode Msx1, Msx2, Msx3, Lmx1a and/or Lmx1b proteins, preferably wherein the Msx1, Msx2, Msx3, Lmx1a and/or Lmx1b are expressed recombinantly, preferably wherein the one or more nucleic acids that encode Msx1, Msx2, Msx3, Lmx1a and/or Lmx1b proteins are introduced by transfection, infection, microinjection, electroporation or recombination, preferably wherein the recombinant expression is effectuated by operably linking the Msx1, Msx2, Msx3, Lmx1a and/or Lmx1b genes to an exogenous promoter and/or enhancer sequence, preferably wherein the exogenous promoter and/or enhancer sequence is contained in an expression vector that is introduced into the cell, preferably wherein the promoter is from the herpes simplex virus thymidine kinase gene or the Rous sarcoma virus LTR, and wherein the enhancer is an enhancer from the nestin gene or a Sonic hedgehog (Shh) enhancer;
optionally wherein the DA neuron is human;
optionally wherein the Msx1, Msx2, Msx3, Lmx1a and/or Lmx1b is encoded by a nucleic acid molecule comprising SEQ ID NO:1 encoding Msx1, SEQ ID NO:3 encoding Msx2, SEQ ID NO:5 encoding Msx3, SEQ ID NO:7 encoding Lmx1a, SEQ ID NO:9 encoding Lmx1b, a coding sequence thereof, or a fragment or derivative thereof that contributes to inducing differentiation of the cell into a DA neuron;
optionally wherein the step of culturing is performed in vitro, in vivo or ex vivo;
optionally wherein the cell is human.

37.-52. (canceled)

53. A method for differentiating a cell to a dopamine neuron in vivo, comprising
ectopically expressing a dopamine neuron determinant in the cell;
optionally wherein the ectopic expression comprises administering to a subject an expression vector that expresses the dopamine neuron determinant;
optionally wherein the cell is an adult neural stem cell;
optionally wherein the dopamine neuron determinant is Msx1, Msx2, Msx3, Lmx1a and/or Lmx1b;
optionally wherein the subject has or is suspected of having Parkinson's disease.

54.-57. (canceled)

58. A method for cell transplantation, comprising
obtaining dopamine (DA) neurons as claimed in claim 19 or as produced by the method of claim 36 or claim 53, and
transplanting the DA neurons into a subject,
optionally wherein the DA neurons are transplanted into the brain of the subject, preferably wherein the DA neurons are transplanted into the striatum.

59.-63. (canceled)

64. A method for treating a neurodegenerative disease or disorder, comprising
obtaining dopamine (DA) neurons as claimed in claim 19 or as produced by the method of any of claim 36 or claim 53, and
transplanting the DA neurons into a subject having or suspected of having the neurodegenerative disease or disorder,
optionally wherein the neurodegenerative disease or disorder is Parkinson's disease;
optionally wherein the DA neurons are transplanted into the brain of the subject, preferably wherein the DA neurons are transplanted into the striatum.

65.-71. (canceled)

72. A method for identifying compounds useful in the differentiation of stem cells, neural and/or neuronal progenitors or precursors to dopamine (DA) neurons, comprising
contacting stem cells, neural and/or neuronal progenitors or precursors with a candidate compound under conditions that, in the absence of the candidate compound, result in a baseline amount of expression of Msx1, Msx2, Msx3, Lmx1a and/or Lmx1b; and
determining a test amount of expression of the Msx1, Msx2, Msx3, Lmx1a and/or Lmx1b in the presence of the candidate compound as a measure of the effect of the compound,
wherein a test amount of expression of the Msx1, Msx2, Msx3, Lmx1a and/or Lmx1b that is greater than the baseline amount indicates that the candidate compound is a compound that is useful in the differentiation of stem cells, neural and/or neuronal progenitors or precursors to DA neurons,
optionally wherein the compound is a set of compounds in a library of molecules, preferably wherein the library is a natural product library, a library generated by combinatorial chemistry or a library of known drug molecules.

73.-76. (canceled)

77. A method for identifying compounds useful in modulating behavior of dopamine (DA) neurons, comprising
contacting DA neurons as claimed in claim 19 with a candidate compound under conditions that, in the absence of the candidate compound, result in a baseline amount of behavior of the DA neurons; and
determining a test amount of behavior of the DA neurons in the presence of the candidate compound as a measure of the effect of the compound,
wherein a test amount of behavior of the DA neurons that is greater than the baseline amount indicates that the candidate compound is a compound that is useful in modulating the behavior of DA neurons,
optionally wherein the compound is a set of compounds in a library of molecules, preferably wherein the library is a natural product library a library generated by combinatorial chemistry or a library of known drug molecules;
optionally wherein the modulating the behavior of DA neurons comprises increasing the growth and/or survival of DA neurons;
optionally wherein the modulating the behavior of DA neurons comprises increasing dopamine synthesis;
optionally wherein the modulating the behavior of DA neurons comprises increasing dopamine storage;
optionally wherein the modulating the behavior of DA neurons comprises increasing dopamine release.

78.-85. (canceled)

86. A method for identifying DA neuron progenitor cells comprising
determining the expression in a cell of one or more Lmx1a, Msx1 and/or Msx2 gene products, wherein the expression of the one or more gene products indicates that the cell is a DA neuron progenitor cell,
optionally wherein the DA neuron progenitor cells are embryonic progenitor cells;
optionally wherein the one or more gene products is RNA, preferably wherein the expression of the one or more gene products is determined by RT-PCR or nucleic acid hybridization;
optionally wherein the one or more gene products is a protein, preferably wherein the expression of the one or more gene products is determined by binding of an antibody or antibody fragment to the protein;
optionally wherein the one or more gene products comprises one or more Lmx1a gene products;
optionally wherein the one or more gene products comprises one or more Msx1 gene products;
optionally wherein the one or more gene products comprises one or more Msx2 gene products.

87.-95. (canceled)

96. A DA neuron progenitor cell identified by the method of claim 86.

97. A method for isolating DA neuron progenitor cells comprising
contacting a population of cells with a reagent that binds to of one or more Lmx1a, Msx1 and/or Msx2 gene products, and
isolating cells that are bound by the reagent from the population,
optionally wherein the DA neuron progenitor cells are embryonic progenitor cells;
optionally wherein the reagent is labeled;
optionally wherein the one or more gene products is a protein, preferably wherein the reagent is an antibody or binding fragment thereof;
optionally wherein the one or more gene products comprises one or more Lmx1a gene products;
optionally wherein the one or more gene products comprises one or more Msx1 gene products;
optionally wherein the one or more gene products comprises one or more Msx2 gene products.

98.-104. (canceled)

105. A DA neuron progenitor cell isolated by the method of claim 97, or a dopamine (DA) neuron differentiated from the DA neuron progenitor cell,
optionally wherein the DA neuron progenitor cell is an embryonic progenitor cell.

106.-107. (canceled)

108. A method of treating a neurodegenerative disease or disorder, comprising
obtaining dopamine (DA) neurons or progenitor cells as claimed in claim 105, and
transplanting the DA neurons or progenitor cells into a subject having or suspected of having the neurodegenerative disease or disorder,
optionally wherein the neurodegenerative disease or disorder is Parkinson's disease;
optionally wherein the DA neurons or progenitor cells are transplanted into the brain of the subject, preferably wherein the DA neurons or progenitor cells are transplanted into the striatum.

109.-111. (canceled)

112. A method for isolating DA neuron progenitor cells and/or DA neurons comprising
providing a population of cells that comprises a nucleic acid molecule that encodes a marker protein operatively linked to a Lmx1a, Msx1 and/or Msx2 promoter sequence, and
isolating cells that express the marker protein from the population,
optionally wherein the DA neuron progenitor cells are embryonic progenitor cells;
optionally wherein the marker protein is a fluorescent protein, preferably wherein the fluorescent protein is a green fluorescent protein;
optionally wherein the marker protein is a cell surface protein;
optionally wherein the cells that express the marker protein are isolated by fluorescence activated cell sorting;
optionally wherein the cells that express the marker protein are isolated by magnetic sorting, preferably wherein the magnetic sorting comprises contacting the population of cells with an antibody or binding fragment thereof that binds to the marker protein, wherein the antibody or binding fragment thereof is linked to a magnetic molecule or particle, and subjecting the population of cells to a magnetic field to separate cells bound by the antibody or binding fragment thereof;
optionally wherein the population of cells comprises stem cells, preferably wherein the stem cells are embryonic stem cells, adult stem cells, or genetically engineered stem cell;
optionally wherein the population of cells are genetically engineered cells.

113.-124. (canceled)

125. A DA neuron progenitor cell or DA neuron isolated by the method of claim 112, or a dopamine (DA) neuron differentiated from the DA neuron progenitor cell.

126. (canceled)

127. A method of treating a neurodegenerative disease or disorder, comprising
obtaining dopamine (DA) neurons or progenitor cells as claimed in claim 125, and
transplanting the DA neurons or progenitor cells into a subject having or suspected of having the neurodegenerative disease or disorder;
optionally wherein the neurodegenerative disease or disorder is Parkinson's disease;
optionally wherein the DA neurons or progenitor cells are transplanted into the brain of the subject, preferably wherein the DA neurons or progenitor cells are transplanted into the striatum.

128-130. (canceled)
